SEQUENCE LISTING (190 sequences)

SEQ ID NO: 1 - protein, 26 aa, Artificial Sequence
  Synthetic Construct
CAALNKKIDDLFLDIWTLNAELLVLL

SEQ ID NO: 2 - protein, 22 aa, Artificial Sequence
  Synthetic Construct
CLNLKNLIEKLKSQIKNLAKEI

SEQ ID NO: 3 - protein, 24 aa, Artificial Sequence
  Synthetic Construct
CAALRGLIGALAGFIEGLWTGIRR

SEQ ID NO: 4 - protein, 24 aa, Artificial Sequence
  Synthetic Construct
CAALTNKINSLIEKINTLFTAIGK

SEQ ID NO: 5 - protein, 24 aa, Artificial Sequence
  Synthetic Construct
CAALGKEIMNLEKRIENLNKKIDD

SEQ ID NO: 6 - protein, 31 aa, Artificial Sequence
  Synthetic Construct
IKSLQNAINGLTNKINSLIEKINTLFTACRR

SEQ ID NO: 7 - protein, 31 aa, Artificial Sequence
  Synthetic Construct
IKSLQNAINRLTNKINSLIEKINTLFTACRR

SEQ ID NO: 8 - protein, 31 aa, Artificial Sequence
  Synthetic Construct
IKSLQNAINRLTNKINTLIEKINTLFTACRR

SEQ ID NO: 9 - protein, 28 aa, Artificial Sequence
  Synthetic Construct
LNKKIDDLFLDIWTLNAELLVLLENCRR

SEQ ID NO: 10 - protein, 24 aa, Artificial Sequence
  Synthetic Construct
ISNLKNLIEKLKSQIKNLAKECRR

SEQ ID NO: 11 - protein, 38 aa, Influenzavirus A
GYAADLKSTQNAIDEITNKVNSVIEKMNTQFTAVGKEF

SEQ ID NO: 12 - protein, 38 aa, Influenzavirus A
GYAADQKSTQNAIDGITNKVNSVIEKMNTQFTAVGKEF

SEQ ID NO: 13 - protein, 38 aa, Influenzavirus A
GYAADQKSTQNAINGITNKVNSIIEKMNTQFTAVGKEF

SEQ ID NO: 14 - protein, 38 aa, Influenzavirus A
GYAADQKSTQNAINGITNKVNTVIEKMNIQFTAVGKEF

SEQ ID NO: 15 - skipped

SEQ ID NO: 16 - protein, 38 aa, Influenzavirus A
GYAADKESTQKAFDGITNKVNSVIEKMNTQFEAVGKEF

SEQ ID NO: 17 - protein, 38 aa, Influenzavirus A
GYAADKESTQKAIDGVTNKVNSIIDKMNTQFEAVGREF

SEQ ID NO: 18 - protein, 36 aa, Influenzavirus A
QAADYKSTQKTIDQVTGKLNRLIEKTNTEFESIESE

SEQ ID NO: 19 - protein, 36 aa, Influenzavirus A
TAADYKSTQSAIDQITGKLNRLIDKTNQQFELIDNE

SEQ ID NO: 20 - protein, 36 aa, Influenzavirus A
TAADLKSTQAAIDQINGKLNRLIEKTNEKYHQIEKE

SEQ ID NO: 21 - skipped

SEQ ID NO: 22 - protein, 36 aa, Influenzavirus A
QAADLKSTQAAIDQINGKLNRLIGKTNEKFHQIEKE

SEQ ID NO: 23 - protein, 38 aa, Influenzavirus A
LEKRIENLNKKVDDGFLDIWTYNAELLVLLENERTLDY

SEQ ID NO: 24 - protein, 38 aa, Influenzavirus A
LEKRIENLNKKVDDGFLDVWTYNAELLVLLENERTLDF

SEQ ID NO: 25 - protein, 38 aa, Influenzavirus A
LEKRMENLNKKVDDGFLDIWTYNAELLVLLENGRTLDF

SEQ ID NO: 26 - protein, 38 aa, Influenzavirus A
LEKRMENLNKKVDDGFLDIWTYNAELLVLLENERTLDF

SEQ ID NO: 27 - protein, 38 aa, Influenzavirus A
LERRIENLNKKVDDGFLDIWTYNAELLVLLENERTLDF

SEQ ID NO: 28 - protein, 38 aa, Influenzavirus A
LERRLENLNKKMEDGFLDVWTYNAELLVLMENERTLDF

SEQ ID NO: 29 - protein, 38 aa, Influenzavirus A
LERRIENLNKKMEDGFLDVWTYNAELLVLMENERTLDF

SEQ ID NO: 30 - protein, 32 aa, Influenzavirus A
EYQIGNVINWTKDSITDIWTYQAELLVAMENQ

SEQ ID NO: 31 - protein, 32 aa, Influenzavirus A
EQQIGNVINWTRDSMTEVWSYNAELLVAMENQ

SEQ ID NO: 32 - protein, 32 aa, Influenzavirus A
EGRIQDLEKYVEDTKIDLWSYNAELLVALENQ

SEQ ID NO: 33-34 - skipped (2 entries)

SEQ ID NO: 35 - protein, 30 aa, Influenzavirus A
DYHDSNVKNLYEKVRSQLKNNAKEIGNGCF

SEQ ID NO: 36 - protein, 30 aa, Influenzavirus A
DFHDSNVKNLYEKVRSQLRNNAKEIGNGCF

SEQ ID NO: 37 - protein, 30 aa, Influenzavirus A
DFHDLNVKNLYEKVKSQLKNNAKEIGNGCF

SEQ ID NO: 38 - protein, 30 aa, Influenzavirus A
DFHDSNVKNLYEKVKSQLKNNAKEIGNGCF

SEQ ID NO: 39 - protein, 30 aa, Influenzavirus A
DFHDSNVRNLYEKVKSQLKNNAKEIGNGCF

SEQ ID NO: 40 - protein, 30 aa, Influenzavirus A
DFHDSNVKNLYDKVRMQLRDNVKELGNGCF

SEQ ID NO: 41 - protein, 30 aa, Influenzavirus A
DFHDSNVKNLYDKVRLQLRDNAKELGNGCF

SEQ ID NO: 42 - protein, 29 aa, Influenzavirus A
DMADSEMLNLYERVRKQLRQNAEEDGKGC

SEQ ID NO: 43 - protein, 29 aa, Influenzavirus A
DLADSEMNKLYERVRKQLRENAEEDGTGC

SEQ ID NO: 44 - protein, 29 aa, Influenzavirus A
DVTDSEMNKLFERVRRQLRENAEDKGNGC

SEQ ID NO: 45 - protein, 29 aa, Influenzavirus A
DVTDSEMNKLFERVRRQLRENAEDQGNGC

SEQ ID NO: 46 - protein, 29 aa, Influenzavirus A
DLTDSEMNKLFEKTKKQLRENAEDMGNGC

SEQ ID NO: 47 - protein, 19 aa, Influenzavirus A
YRGLFGAIAGFIEGGWTGM

SEQ ID NO: 48-53 - skipped (6 entries)

SEQ ID NO: 54 - protein, 19 aa, Influenzavirus A
SRGLFGAIAGFIEGGWTGM

SEQ ID NO: 55-57 - skipped (3 entries)

SEQ ID NO: 58 - protein, 19 aa, Influenzavirus A
SRGLFGAIAGFIEGGWQGM

SEQ ID NO: 59 - protein, 19 aa, Influenzavirus A
KRGLFGAIAGFIEGGWQGM

SEQ ID NO: 60 - protein, 21 aa, Influenzavirus A
ITNKVNSVIEKMNTQFTAVGK

SEQ ID NO: 61-68 - skipped (8 entries)

SEQ ID NO: 69 - protein, 21 aa, Influenzavirus A
ITNKVNTVIEKMNIQFTAVGK

SEQ ID NO: 70 - skipped

SEQ ID NO: 71 - protein, 21 aa, Influenzavirus A
ITNKVNSVIEKMNTQFEAVGK

SEQ ID NO: 72 - protein, 21 aa, Influenzavirus A
VTNKVNSIINKMNTQFEAVGR

SEQ ID NO: 73 - protein, 21 aa, Influenzavirus A
VGKEFNNLEKRMENLNKKVDD

SEQ ID NO: 74-79 - skipped (6 entries)

SEQ ID NO: 80 - protein, 21 aa, Influenzavirus A
VGKEFNHLEKRIENLNKKVDD

SEQ ID NO: 81 - skipped

SEQ ID NO: 82 - protein, 21 aa, Influenzavirus A
VGKEFNKLEKRMENLNKKVDD

SEQ ID NO: 83 - protein, 21 aa, Influenzavirus A
VGKEFNNLERRIENLNKKVDD

SEQ ID NO: 84 - protein, 21 aa, Influenzavirus A
VGKEFSNLERRIENLNKKMED

SEQ ID NO: 85 - protein, 20 aa, Influenzavirus A
VGREFNNLERRIENLNKKME

SEQ ID NO: 86 - protein, 23 aa, Influenzavirus A
LNKKVDDGFLDIWTYNAELLVLL

SEQ ID NO: 87-93 - skipped (7 entries)

SEQ ID NO: 94 - protein, 23 aa, Influenzavirus A
LNKKVDDGFLDVWTYNAELLVLM

SEQ ID NO: 95-96 - skipped (2 entries)

SEQ ID NO: 97 - protein, 23 aa, Influenzavirus A
LNKKMEDGFLDVWTYNAELLVLM

SEQ ID NO: 98 - protein, 23 aa, Influenzavirus A
LNKKMEDGFLDVWTYNAELLVLL

SEQ ID NO: 99 - protein, 22 aa, Influenzavirus A
DLNVKNLYEKVKSQLKNNAKEI

SEQ ID NO: 100-105 - skipped (6 entries)

SEQ ID NO: 106 - protein, 22 aa, Influenzavirus A
DSNVKNLYEKVRSQLKNNAKEI

SEQ ID NO: 107 - protein, 22 aa, Influenzavirus A
DSNVKNLYEKVRSQLRNNAKEI

SEQ ID NO: 108 - protein, 22 aa, Influenzavirus A
DSNVKNLYEKVRSQLRNNAKEI

SEQ ID NO: 109 - protein, 22 aa, Influenzavirus A
DSNVRNLYEKVKSQLKNNAKEI

SEQ ID NO: 110 - protein, 22 aa, Influenzavirus A
DSNVKNLYDKVRMQLRDNVKEL

SEQ ID NO: 111 - protein, 22 aa, Influenzavirus A
DSNVKNLYDKVRMQLRDNAKEL

SEQ ID NO: 112 - protein, 31 aa, Influenzavirus A
QKSTQNAINRITNKVNSIIEKMNTQFTAVGK

SEQ ID NO: 113 - protein, 31 aa, Influenzavirus A
QKSTQNAINGITNKVNSIIEKMNTQFTAVGK

SEQ ID NO: 114-118 - skipped (5 entries)

SEQ ID NO: 119 - protein, 31 aa, Influenzavirus A
LKSTQNAINEITNKVNSVIEKMNTQFTAVGK

SEQ ID NO: 120 - protein, 31 aa, Influenzavirus A
QKSTQNAIDGITNKVNSVIEKMNTQFTAVGK

SEQ ID NO: 121 - protein, 31 aa, Influenzavirus A
QKSTQNAINGITNKVNTVIEKMNIQFTAVGK

SEQ ID NO: 122 - skipped

SEQ ID NO: 123 - protein, 31 aa, Influenzavirus A
KESTQKAFDGITNKVNSVIEKMNTQFEAVGK

SEQ ID NO: 124 - protein, 31 aa, Influenzavirus A
KESTQKAIDGITNKVNSIIENMNTQFEAVGR

SEQ ID NO: 125 - protein, 29 aa, Influenzavirus A
QKSTQNAINGITNKVNTVIEKMNIQFTAV

SEQ ID NO: 126-131 - skipped (6 entries)

SEQ ID NO: 132 - protein, 31 aa, Influenzavirus A
LKSTQNAINEITNKVNTVIEKMNIQFTAVGK

SEQ ID NO: 133 - protein, 31 aa, Influenzavirus A
QKSTQNAIDGITNKVNTVIEKMNIQFTAVGK

SEQ ID NO: 134 - protein, 31 aa, Influenzavirus A
QKSTQNAINGITNKVNSVIEKMNTQFTAVGK

SEQ ID NO: 135 - skipped

SEQ ID NO: 136 - protein, 31 aa, Influenzavirus A
KESTQKAFDGITNKVNTVIEKMNIQFEAVGK

SEQ ID NO: 137 - protein, 31 aa, Influenzavirus A
KESTQKAIDGVTNKVNTIINKMNIQFEAVGR

SEQ ID NO: 138 - protein, 29 aa, Influenzavirus A
MENLNKKVDDGFLDIWTYNAELLVLLENE

SEQ ID NO: 139 - skipped

SEQ ID NO: 140 - protein, 31 aa, Artificial Sequence
  Synthetic Construct
IENLNKKIDDLFLDIWTLNAEILVLLENCRR

SEQ ID NO: 141-144 - skipped (4 entries)

SEQ ID NO: 145 - protein, 29 aa, Influenzavirus A
IENLNKKVDDGFLDIWTYNAELLVLLENE

SEQ ID NO: 146 - protein, 29 aa, Influenzavirus A
IENLNKKVDDGFLDVWTYNAELLVLLENE

SEQ ID NO: 147 - protein, 29 aa, Influenzavirus A
MENLNKKVDDGFLDIWTYNAELLVLLENG

SEQ ID NO: 148 - skipped

SEQ ID NO: 149 - protein, 29 aa, Influenzavirus A
LENLNKKMEDGFLDVWTYNAELLVLMENE

SEQ ID NO: 150 - protein, 29 aa, Influenzavirus A
IENLNKKMEDGFLDVWTYNAELLVLMENE

SEQ ID NO: 151 - protein, 29 aa, Influenzavirus A
ERTLDFHDSNVKNLYEKVKSQLKNNAKEI

SEQ ID NO: 152 - skipped

SEQ ID NO: 153 - protein, 31 aa, Artificial Sequence
  Synthetic Construct
IRTLDFHISNLKNLIEKLKSQIKNLAKECRR

SEQ ID NO: 154-157 - skipped (4 entries)

SEQ ID NO: 158 - protein, 29 aa, Influenzavirus A
ERTLDYHDSNVKNLYEKVRSQLKNNAKEI

SEQ ID NO: 159 - protein, 29 aa, Influenzavirus A
ERTLDFHDSNVKNLYEKVRSQLRNNAKEI

SEQ ID NO: 160 - protein, 30 aa, Influenzavirus A
EGRTLDFHDLNVKNLYEKVKSQLKNNAKEI

SEQ ID NO: 161 - protein, 29 aa, Influenzavirus A
ERTLDFHDSNVRNLYEKVKSQLKNNAKEI

SEQ ID NO: 162 - protein, 29 aa, Influenzavirus A
ERTLDFHDSNVKNLYDKVRMQLRDNVKEL

SEQ ID NO: 163 - protein, 29 aa, Influenzavirus A
ERTLDFHDSNVKNLYDKVRMQLRDNAKEL

SEQ ID NO: 164 - protein, 58 aa, Influenzavirus A
GWYGYHHQNEQGSCGYAADLKSTQNAIDEITNKVNSVIEKMNTQFTAVGKEFNHLEKR

SEQ ID NO: 165 - protein, 58 aa, Influenzavirus A
GWYGYHHQNGQGSCGYAADQKSTQNAIDGITNKVNSVIEKMNTQFTAVGKEFNHLEKR

SEQ ID NO: 166 - protein, 58 aa, Influenzavirus A
GWYGYHHQNEQGSCGYAADQKSTQNAINGITNKVNSIIEKMNTQFTAVGKEFNNLERR

SEQ ID NO: 167 - protein, 58 aa, Influenzavirus A
GWYGYHHQNEQGSCGYAADQKSTQNAINGITNKVNTVIEKMNIQFTAVGKEFNNLEKR

SEQ ID NO: 168 - protein, 58 aa, Influenzavirus A
GWYGYHHQNEQGSCGYAADQKSTQNAIDGITNKVNSVIEKMNTQFTAVGKEFNKLEKR

SEQ ID NO: 169 - protein, 58 aa, Influenzavirus A
GWYGYHHSNDQGSCGYAADKESTQKAFDGITNKVNSVIEKMNTQFEAVGKEFSNLERR

SEQ ID NO: 170 - protein, 58 aa, Influenzavirus A
GWYGYHHSNEQGSCGYAADKESTQKAIDGVTNKVNSIINKMNTQFEAVGREFNNLERR

SEQ ID NO: 171 - protein, 61 aa, Influenzavirus A
IENLNKKVDDGFLDIWTYNAELLVLLENERTLDYHDSNVKNLYEKVRSQLKNNAKEIGNGC

SEQ ID NO: 172 - protein, 61 aa, Influenzavirus A
IENLNKKVDDGFLDVWTYNAELLVLLENERTLDFHDSNVKNLYEKVRSQLRNNAKEIGNGC

SEQ ID NO: 173 - protein, 61 aa, Influenzavirus A
IENLNKKVDDGFLDIWTYNAELLVLLENERTLDFHDSNVRNLYEKVKSQLKNNAKEIGNGC

SEQ ID NO: 174 - protein, 61 aa, Influenzavirus A
MENLNKKVDDGFLDIWTYNAELLVLLENGRTLDFHDLNVKNLYEKVKSQLKNNAKEIGNGC

SEQ ID NO: 175 - protein, 61 aa, Influenzavirus A
MENLNKKVDDGFLDIWTYNAELLVLLENERTLDFHDSNVKNLYEKVKSQLKNNAKEIGNGC

SEQ ID NO: 176 - protein, 61 aa, Influenzavirus A
LENLNKKMEDGFLDVWTYNAELLVLMENERTLDFHDSNVKNLYDKVRMQLRDNVKELGNGC

SEQ ID NO: 177 - protein, 61 aa, Influenzavirus A
IENLNKKMEDGFLDVWTYNAELLVLMENERTLDFHDSNVKNLYDKVRLQLRDNAKELGNGC

SEQ ID NO: 178 - protein, 31 aa, Artificial Sequence
  Synthetic Construct
  VARIANT 5, 6, 7, 9, 10, 12, 13, 14, 16, 17, 19, 20, 21, 23, 24,26, 27, 28: Xaa = Any Viral Amino Acid
CAALXXXIXXLXXXIXXLXXXIXXLXXXIRR

SEQ ID NO: 179 - skipped

SEQ ID NO: 180 - protein, 21 aa, Influenzavirus A
ITNKVNSIIEKMNTQFTAVGK

SEQ ID NO: 181-188 - skipped (8 entries)

SEQ ID NO: 189 - protein, 29 aa, Influenzavirus A
QKSTQNAINGITNKVNTVIEKMNIQFTAY

SEQ ID NO: 190 - protein, 31 aa, Artificial Sequence
  Synthetic Construct
IKSLQNAINGLTNKINTLIEKINILFTACRR

(19) United States
(12) Patent Application Publication
(10) Pub. No.: US 2012/0014972 A1
(43) Pub. Date: Jan. 19, 2012

(54) INFLUENZA VIRUS COMPOSITIONS AND METHODS FOR UNIVERSAL VACCINES

(75) Inventors: Robert S. Hodges, Denver, CO (US); Brooke Elizabeth Bishop Hirsch, Broomfield, CO (US); Zhe Yan, Lakewood, CO (US); Kathryn V. Holmes, Golden, CO (US); Zhaohui Qian, Colorado Springs, CO (US); Wendy Hartsock, Aurora, CO (US)

(73) Assignee: THE REGENTS OF THE UNIVERSITY OF COLORADO, A BODY CORPORATE

(21) Appl. No.: 13/014,682

(22) Filed: Jan. 26, 2011

Related U.S. Application Data
(60) Provisional application No. 61/298,354, filed on Jan. 26, 2010.

(51) Int. Cl. A61K 39/145; C07K 16/10; A61P 31/16; A61K 39/42; C07K 19/00; C07K 17/02; C07K 16/46

(52) U.S. Cl. 424/159.1; 530/324; 424/210.1; 530/389.4; 530/387.3; 530/388.3; 424/196.11; 530/323

(57) ABSTRACT

The disclosure relates at least in part to embodiments of compositions and methods including vaccines for protection against multiple serologically distinct strains of influenza virus. This disclosure provides significant advances and addresses important needs in the influenza vaccine field.

### CROSS-REFERENCE TO RELATED APPLICATIONS

This application claims priority benefit of U.S. Provisional Patent Application No. 61/298,354, filed Jan. 26, 2010. The entire contents of that application are hereby incorporated by reference herein.

### STATEMENT ON FEDERALLY SPONSORED RESEARCH OR DEVELOPMENT

Not applicable

### BACKGROUND

Influenza remains a pandemic disease that infects hundreds of millions of people annually. Every decade or so, an antigenically distinct strain of influenza A virus emerges in animals and spreads to humans where it inflicts widespread disease, misery, economic loss, and death. The current approaches to influenza A virus vaccines and therapeutic treatments do not adequately address the many problems. These problems relate in part to the diverse and changing attributes of the genome, gene expression products, and antigenic determinants of the various and newly emerging virus strain(s). Influenza A viruses constantly undergo antigenic drift, a process of mutation leading to changed antigenic epitopes, as they move through a partially immune human population. Also, new strains with novel hemagglutinin (HA) and/or neuraminidase (NA) glycoprotein antigens are occasionally introduced into humans from animal reservoirs and adapt to human-to-human spread in a process called antigenic shift. These changed and new strains can exhibit an increased level of virulence and/or rapid transmission in a population with immune systems which are relatively unfamiliar with the new antigens. Existing vaccine approaches in particular suffer from the disadvantage of always lagging behind the appearance of the new antigenically distinct influenza A virus strains. Embodiments of the present invention are directed to novel vaccine compositions and methods to protect against many serologically distinct strains of influenza virus. This disclosure provides significant advances and addresses important needs in the influenza vaccine field.

### SUMMARY OF THE INVENTION

In general the terms and phrases used herein have their art-recognized meaning, which can be found by reference to standard texts, journal references and contexts known to those skilled in the art. The following definitions are provided to clarify their specific use in the context of the invention.

In the context of certain vaccine embodiments, “broadly protective” refers to the ability to induce protection against different influenza viruses, e.g., against multiple, serologically distinct influenza virus strains. A “neutralizing antibody” is understood in the art and for certain examples refers to immunoglobulin from a host animal which is capable of preventing or inhibiting virus infection. For certain embodiments when discussing hemagglutinin glycoprotein structure, the “stem region” is pertinent to the HA₂ domain of the influenza HA protein.

The following abbreviations are applicable. HA, hemagglutinin glycoprotein; NA, neuraminidase glycoprotein; TT, tetanus toxoid; BSA, bovine serum albumin; KLH, keyhole limpet hemocyanin.

Embodiments of the invention relate to advances in compositions and methods for the prevention and treatment of influenza virus infections and disease.

In an embodiment, the invention provides a conjugate of a conformationally stabilized two-stranded peptide unit and a carrier molecule, the conjugate having the structural formula FX1:

CX-LX3-{[LX2]-[PX1-LX1-PX2]}ₘ (FX1);

wherein
PX1 is a first synthetic peptide,
PX2 is a second synthetic peptide,
LX1 is a first linker for covalently linking the first peptide to the second peptide;
wherein PX1-LX1-PX2 form a conformationally stabilized two-stranded peptide unit;
LX2 is a second linker for linking the two-stranded peptide unit to a carrier molecule,
LX3 is a third linker or direct bond for linking the carrier molecule to the second linker LX2,
CX is an immunogenic carrier molecule, and
m is an integer greater than or equal to one;
wherein the PX1 and PX2 synthetic peptides each independently comprise an adapted peptide sequence corresponding to a stem region of an influenza virus hemagglutinin (HA) protein, wherein the adapted sequence consists of a native or synthetic HA₂ domain segment of 15 to 40 amino acids which is integrated in a coiled-coil template, and wherein the conformationally stabilized two-stranded peptide unit, PX1-LX1-PX2 comprises an alpha-helical structure. In an embodiment, the PX1-LX1-PX2 component comprises a coiled-coil structure. In an embodiment, the domain segment is 15, 16, 17, 18, 19, 20, 21, 22, 23, 24, 25, 26, 27, 28, 29, 30, 31, 32, 33, 34, 35, 36, 37, 38, 39, or 40 amino acids in length.

In an embodiment, the invention provides a conjugate wherein the influenza virus hemagglutinin HA₂ domain segments of first peptide PX1 and second peptide PX2 are each independently a segment derived from the native or synthetic HA₂ domain segment corresponding to at least one of the sequence of amino acid residues for peptides: P3, amino acids 391-411; P4, 409-429; P5, 423-445; P6, 455-476; 1A, WSN HA (342-360); 3A, WSN HA (391-411); 4A, WSN HA (409-429); 5A, WSN HA (423-445); 6A, WSN HA (455-476); 3M1 [also referred to as 3M], WSN HA (381-411); 3M2 [also referred to as 3M*], WSN HA (381-411); 3 MP, PR8 HA (381-409); 5P, PR8 HA (423-448); 6P, PR8 HA (455-476); or a variation thereof; where residue numbering is relative to a full sequence for an H3N2 influenza virus strain.

In embodiments, the two arginine residues (R) are optionally added, e.g., to enhance solubility of the synthetic peptides and conjugate. Thus in various embodiments a given sequence relevant to a hemagglutinin peptide segment is provided where the sequence is flanked by RR to indicate the arginine residues. Therefore, in embodiments the peptide components PX1 and PX2 can each individually be considered to further include an RR component.

In embodiments, a component selected from the group consisting of -Arg, -(Arg)₂, -(Arg)₃, -(Arg)₄, -Lys, -(Lys)₂, -(Lys)₃, -(Lys)₄, -Arg-amide, -(Arg)₂-amide, -(Arg)₃-amide, -(Arg)₄-amide, -Lys-amide, -(Lys)₂-amide, -(Lys)₃-amide, -(Lys)₄-amide, -Cys-Arg, -Cys-(Arg)₂, -Cys-(Arg)₃, -Cys-(Arg)₄, -Cys-Lys, -Cys-(Lys)₂, -Cys-(Lys)₃, -Cys-(Lys)₄, -Cys-Arg-amide, -Cys-(Arg)₂-amide, -Cys-(Arg)₃-amide, -Cys-(Arg)₄-amide, -Cys-Lys-amide, -Cys-(Lys)₂-amide, -Cys-(Lys)₃-amide, and -Cys-(Lys)₄-amide is optionally added to the C-terminus of one or both the peptide components PX1 and PX2. Thus in various embodiments a given sequence relevant to a hemagglutinin peptide segment is provided where the sequence is flanked by one of these components. Therefore, in embodiments the peptide components PX1 and PX2 can each individually be considered to further include one of these components.

In an embodiment, the invention provides a conjugate wherein the adapted peptide sequences for PX1 and PX2 are each independently at least one of the sequences:

5A.T,

| | |
|---|---|
| CAALNKKIDDLFLDIWTLNAELLVLL; | (SEQ ID NO: 1) |
| 6A.T, | |
| CLNLKNLIEKLKSQIKNLAKEI; | (SEQ ID NO: 2) |
| 1A.T, | |
| CAALRGLIGALAGFIEGLWTGIRR; | (SEQ ID NO: 3) |
| 3A.T, | |
| CAALTNKINSLIEKINTLFTAIGK; | (SEQ ID NO: 4) |
| 4A.T, | |
| CAALGKEIMNLEKRIENLNKKIDD; | (SEQ ID NO: 5) |
| 3M1.T/3M.T, | |
| IKSLQNAINGLTNKINSLIEKINTLFTACRR; | (SEQ ID NO: 6) |
| 3M2.T/3M*.T, | |
| IKSLQNAINRLTNKINSLIEKINTLFTACRR; | (SEQ ID NO: 7) |
| 3MP.T, | |
| IKSLQNAINRLTNKINTLIEKINTLFTACRR; | (SEQ ID NO: 8) |
| 5P.T, | |
| IENLNKKIDDLFLDIWTLNAEILVLLENCRR; | (SEQ ID NO: 140) |
| 6P.T, | |
| IRTLDFHISNLKNLIEKLKSQIKNLAKECRR; | (SEQ ID NO: 153) |
| 5P.T_alt, | |
| LNKKIDDLFLDIWTLNAELLVLLENCRR; | (SEQ ID NO: 9) |
| 6P.T_alt, | |
| ISNLKNLIEKLKSQIKNLAKECRR; | (SEQ ID NO: 10) |

or a variation thereof. In certain instances the “.T” designation refers to a templated peptide sequences, while the “_alt” designation refers to an alternative sequence.

In an embodiment, the invention provides a peptide compound or conjugate wherein the variation is such that a variant sequence is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identical to a reference sequence. In an embodiment, the variation is a conservative substitution or permits from 1 to 5 changes relative to a reference sequence.

In an embodiment, the coiled-coil template has a peptide sequence with an isoleucine residue at an “a” position and a leucine reside at a “d” position of a heptad helical unit in at least two heptad units corresponding to the HA₂ domain segment. In an embodiment, the template is a template as described in U.S. Pat. No. 6,872,806 or according to Lu and Hodges, 2002 (J Biol Chem 277:23515-24). In an embodiment, a peptide segment of a peptide, e.g., a PX1 or PX2 peptide, comprises a repeating heptad sequence (a b c d e f g)ₙ. In a particular embodiment, n equals from 2 to 10. In a preferred embodiment, n is at least two. In a preferred embodiment, n is 2, 3, 4, 5, or 6.

In an embodiment, the conformationally stabilized, templated two-stranded peptide unit is capable of forming one or more epitopes which mimic the structure of a region in the stem of one or more native hemagglutinin molecules in its pre-fusion conformation.

In an embodiment, the first and second peptides PX1 and PX2 each have the same peptide sequence, and the unit is referred to as a homo two-stranded peptide combination or conjugate. In an embodiment, the first and second peptides PX1 and PX2 are different peptides. In an embodiment where first and second peptides PX1 and PX2 are different peptides, the combination or conjugate may be referred to as a hetero two-stranded templated combination or conjugate. In an embodiment, a hetero two-stranded coiled-coil contains two different epitopes. In a preferred hetero two-stranded conjugate embodiment, the length of each alpha helix is the same and the “a” and “d” positions are in-register (i.e., “a” of one peptide matching with “a” of the other peptide, and likewise for “d”). In an embodiment, a hetero two-stranded conjugate is the basis for a multi-epitope synthetic peptide conjugate. For example, such a hetero two-stranded templated conjugate can be a single immunogen containing at least two distinct epitopes, comprised of at least two distinct epitopes derived from different alpha helical regions of the stem of the same influenza HA protein in its pre-fusion conformation.

In an embodiment of a hetero two-stranded conjugate, the invention provides a conjugate, wherein the first and second peptides PX1 and PX2 are selected from the group consisting of: a first pair of 3 MP PR8 HA₂ (381-409) (SEQ ID NO: 198) and 5P PR8 HA₂ (420-448) (SEQ ID NO:199); a second pair of 3 MP PR8 HA₂ (381-409) (SEQ ID NO:202) and 6P PR8 HA₂ (448-476) (SEQ ID NO:203); and a third pair of 5P PR8 HA₂ (420-448) (SEQ ID NO:206) and 6P PR8 HA₂ (448-476) (SEQ ID NO:207); wherein for each member of each pair a templated sequence is provided in FIG. 14 and optionally includes a flanking RR component. Additional pairs and variants are contemplated in further embodiments according to teachings herein.

In an embodiment, the invention provides a conformationally stabilized two-stranded peptide compound, the compound having the structural formula FX2:

[PX1-LX1-PX2] (FX2);

wherein
PX1 is a first synthetic peptide,
PX2 is a second synthetic peptide,
LX1 is a first linker for covalently linking the first peptide to the second peptide;
wherein PX1-LX1-PX2 form a conformationally stabilized two-stranded peptide unit;
wherein the PX1 and PX2 synthetic peptides each independently comprise an adapted peptide sequence corresponding to a stem region of an influenza virus hemagglutinin protein, wherein the adapted sequence consists of a native or synthetic HA₂ domain segment of 15 to 40 amino acids which is integrated in a coiled-coil template, and wherein the conformationally stabilized two-stranded peptide unit, PX1-LX1-PX2 comprises an alpha-helical structure.

In an embodiment, the invention provides a compound further comprising a second linker LX2, where the compound has structural formula FX3:

[LX2]-[PX1-LX1-PX2] (FX3);

wherein
LX2 is a second linker for linking the two-stranded peptide unit to a carrier molecule, for example, a macromolecule such as a protein, or a substrate. In one embodiment, LX2 is attached to the N-terminus of PX1.

In an embodiment, the invention provides a composition comprising a conjugate or compound in a pharmaceutically acceptable formulation. In an embodiment, the invention provides a composition comprising a conjugate or compound and an adjuvant.

In one embodiment of the invention, the carrier molecule of the conjugate is a protein. The protein can be keyhole limpet hemocyanin (KLH), bovine serum albumin (BSA), ovalbumin, tetanus toxoid, cholera subunit B, protein D from H. influenza, or diphtheria toxoid. In another embodiment of the invention, the carrier molecule of the conjugate is a non-proteinaceous moiety. The non-proteinaceous moiety can be a polysaccharide, such as alginic acid (alginate). In a further embodiment, the carrier molecule is selected from the group consisting of keyhole limpet hemocyanin (KLH), tetanus toxoid (TT), and bovine serum albumin (BSA).

In an embodiment, the invention provides a method of inducing an immune response against influenza virus HA protein, comprising contacting a mammal with a composition, conjugate or compound described herein. In an embodiment, the invention provides a method of preventing an influenza virus infection or attenuating the virulence of an influenza virus infection, comprising administering to a subject an effective amount of a composition, conjugate or compound described herein.

In an embodiment, the invention provides a molecule which is an antibody, fragment thereof, or other antigen recognition molecule capable of binding to a conjugate or compound herein, wherein the binding is to an epitope of the conformationally stabilized templated two-stranded peptide unit. In an embodiment, the IgG molecule or fragment thereof is humanized or fully human. In an embodiment, the molecule is a monoclonal antibody. In an embodiment, the molecule is a part of a polyclonal composition of such molecules. In an embodiment, the molecule is capable of neutralizing an influenza virus. In an embodiment, the molecule is capable of inhibiting the infectivity of an influenza virus in vitro. In an embodiment, the molecule is capable of inhibiting the infectivity of an influenza virus in vivo. In an embodiment, the invention provides a method of therapy for an influenza infection comprising administering an effective amount of the molecule to a subject in need thereof.

In an embodiment, the invention provides a use of a conjugate, compound, or composition herein in the manufacture of a medicament. In an embodiment, the invention provides a use of a conjugate, compound, or composition herein in the manufacture of a medicament for the prevention or treatment of an influenza virus infection. In an embodiment, the invention provides a use of a conjugate, compound, or composition herein for the prevention or treatment of an influenza virus infection.

In an embodiment, the linker LX1 occurs between residues located at the N-terminus of PX1 and PX2, or between residues located at the C-terminus of PX1 and PX2.

In an embodiment, the invention provides a conjugate or compound wherein the linker LX1 is a disulfide bridge between sulfur-containing amino acid residues of PX1 and PX2.

In an embodiment, the invention provides a conjugate or compound wherein the linker LX1 is a compound of the form R₁(—NH₂)—R₂-R₃(—NH₂), where R₁ and R₃ can independently be C₁-C₈ hydrocarbyl, C₁-C₈ alkyl, C₁-C₈ heteroalkyl, HOOC—C₁-C₈ hydrocarbyl, or HOOC—C₁-C₈ alkyl, and R₂ can be C₁-C₈ hydrocarbylene, C₁-C₈ alkylene, C₁-C₈ heteroalkylene (preferably C₁-C₈ alkylene) or a nonentity. In another embodiment, the linker LX1 is HOOC—(CH₂)ₓ—CH(NH₂)—(CH₂)_{y}—CH(NH₂)—(CH₂)_{z}—H, where x, y, and z are independently of each other integers between 0 and 6, inclusive. In another embodiment, the linker LX1 is 2,3,-diaminopropionic acid. In these embodiments, the linker LX1 is attached to the C-terminus of PX1 and PX2 by a C-terminal amide bond between PX1 and one of the amino groups of LX1, and a C-terminal amide bond between PX2 and the other amino group of LX1.

In embodiments, the two-stranded, conformation-stabilized, coiled-coil peptide immunogens elicit antibodies to specific alpha-helical epitopes in the influenza HA2 region that lock the native protein in its pre-fusion conformation, affecting membrane fusion events and thereby reducing or preventing efficient viral infection and disease. In one embodiment, the antibodies affect the membrane fusion event by reducing or inhibiting membrane fusion events.

In an embodiment, the conjugates of the invention are isolated or purified.

In an embodiment, a composition of the invention is a peptide compound.

In an embodiment, the invention provides a pharmaceutical formulation comprising a composition of the invention, such as a conjugate of the invention. In an embodiment, the invention provides a method of synthesizing a composition of the invention or a pharmaceutical formulation thereof. In an embodiment, a pharmaceutical formulation comprises one or more excipients, carriers, and/or other components as would be understood in the art. In an embodiment, an effective amount of a composition of the invention can be a therapeutically effective amount.

In an embodiment, a peptide composition of the invention is prepared using recombinant methodology or synthetic techniques. In an embodiment, a nucleic acid composition of the invention is prepared using recombinant methodology or synthetic techniques.

In an embodiment, the invention provides a method for treating a medical condition comprising administering to a subject in need thereof, a therapeutically effective amount of a composition of the invention, such as a conjugate of the invention. In an embodiment, the medical condition is influenza disease.

In an embodiment, the invention provides a medicament which comprises an effective amount, for example a therapeutically effective amount, of one or more compositions or conjugates of the invention. In an embodiment, the invention provides a method for making a medicament for treatment of a condition described herein.

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE FIGURES

FIG. 1 illustrates an influenza virion. Neutralizing antibody is bound to the receptor-binding domain on trimeric hemagglutinin (HA) glycoproteins. The tetrameric neuraminidase (NA) glycoprotein is also inserted in the viral envelope. Eight genome segments are depicted in the virion (public domain image obtained from the US National Institute of Allergy and Infectious Diseases (NIAID) according to niaid.nih.gov/topics/Flu/Research/basic/).

FIG. 2 illustrates phylogenetic relationships among the 16 subtypes of HA in influenza A viruses of humans, animals and birds.

FIG. 3 illustrates a model for HA₂-mediated fusion of influenza A viral envelope with host membrane (top). Helical regions in trimeric HA₂ are show as colored cylinders. FIG. 3A indicates the pre-fusion conformation of the HA₂ domain of influenza HA. At acid pH and 37° C., the HA₂ region refolds to extend the hydrophobic fusion peptide (red) into the host cell membrane (steps 2 to 4). A second major conformational change in HA₂ (steps 4 to 6) draws the host and viral membranes together to form the fusion pore (last step), allowing the viral genome segments or nucleocapsids to enter the cytoplasm to begin the replicative cycle.

FIG. 4 illustrates a strategy for preparation of influenza-based template-carrier protein conjugates. The residue positions in the template that are substituted with the native sequence of an influenza hemagglutinin segment are indicated with an asterisk. Residues forming the 4-3/3-4 hydrophobic repeat for two-stranded, alpha-helical coiled-coil formation are underlined. The positions “a” and “d” are denoted in the context of the repeating heptad sequence (a b c d e f g)n. In cross-section view, the direction of the helices is into the page from N- to C-terminus with polypeptide chains parallel and in-register. Arrows denote the hydrophobic interactions between residues a and a′ and d and d′ in the hydrophobic core. In the schematic model, shaded circles indicate the substituted positions on the front helix of the disulfide bridged two-stranded coiled-coil immunogen.

FIG. 5 illustrates generation of the templated peptide immunogen. In the disulfide-bridged two-stranded template, one strand contains the photo-activated crosslinker (BB, benzoyl-benzoyl), norleucine (nL) to determine the peptide to carrier ratio after coupling, and a flexible glycine residue between the linker and peptide. DTNP, dithionitropyridine, is used for formation of a 1:1 disulfide-bridged peptide. The two-stranded template is coupled to a carrier protein (KLH or another appropriate protein for immunization or BSA, etc., for antibody evaluations).

FIG. 6 illustrates the trimeric HA spike of influenza virus in its pre-fusion conformation and certain epitopes. FIG. 6A) shows epitopes 3, 5, and 6 in helical or partially helical regions of the stem of the WSN strain of influenza A H1. The structure is for the mouse-adapted strain A/PR/8/24. Relevant to these epitopes, the conformation-stabilized multi-stranded peptide immunogens 3A, 5A, and 6A are shown in FIG. 6B). In the center panel, a space-filling model in the same orientation shows that the epitopes are exposed on the surface of the stem region.

FIG. 7 illustrates results of experiments testing the protection conferred by antibodies arising from immunization with the peptide conjugates. Rabbit antibody preparations were generated to the peptide conjugates and used for passive immunization of mice. The mice were subsequently challenged intranasally with mouse-adapted influenza virus. The y-axes show percent survival over time x-axes). Representative data are portrayed from two independent experiments.

FIG. 8 illustrates aspects of the sequences in epitopes 3, 5, and 6 in the stem of influenza A H1, H2, and H5 proteins. The epitopes are shown in boxes. Identical residues are highlighted in green, conserved residues, in gray, and non-conserved residues, in white. Epitope 3 includes the entire domain designated Helix A. To the right of the vertical dashed yellow line (and inverted triangle) are residues for the segment relevant to peptide immunogen 3A. The immunogen 3M includes all of the helix A/epitope 3 segment. Helix A is described by Sui et al., 2009 (Nat Struct Mol Biol 16:265-73), including at FIG. 4 therein where it is referred to as the αA helix.

FIG. 9 illustrates selected surface-exposed, epitopes and alpha-helical peptides of influenza HA protein. Yellow boxes indicate the two-stranded, conformation-stabilized, templated, coiled-coil peptides for conjugation to carrier proteins.

FIG. 10 illustrates the generation of TT-coupled influenza peptide-based immunogens.

FIG. 11 illustrates structure of the exodomain of the influenza virus HA protein and certain peptides. The fusion monomer depicted in (A) has 138 residues, and the fusion trimer in (B) has 414 total residues. P3=residue 391-411; P4=residue 409-429; P5=residue 423-445; P6=residue 455-476 (residue numbers based on H3N2 full sequence).

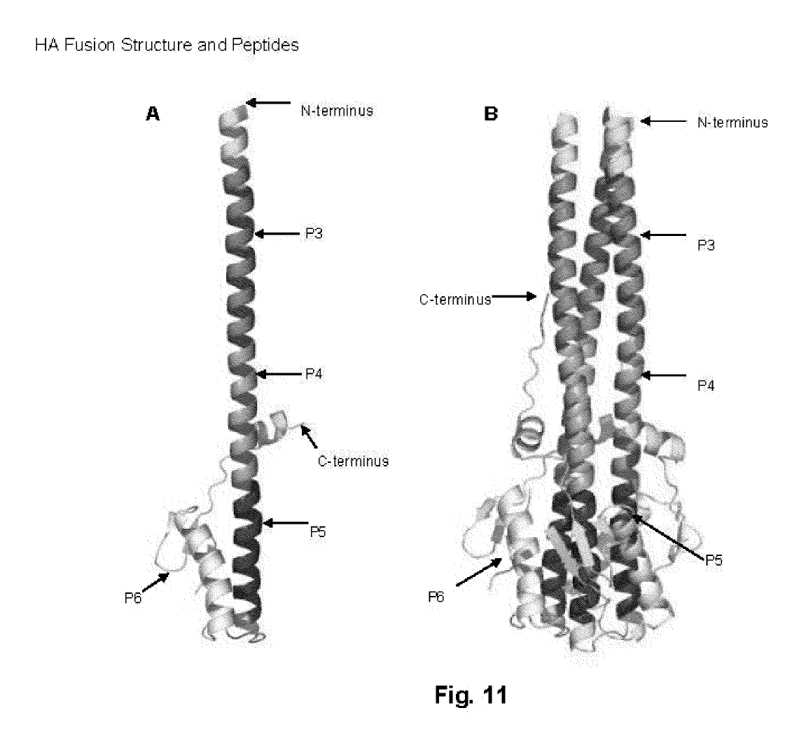

FIG. 12 illustrates the exodomain of the HA₀ pre-fusion structure and certain peptides. The pre-fusion molecule has 494 residues.

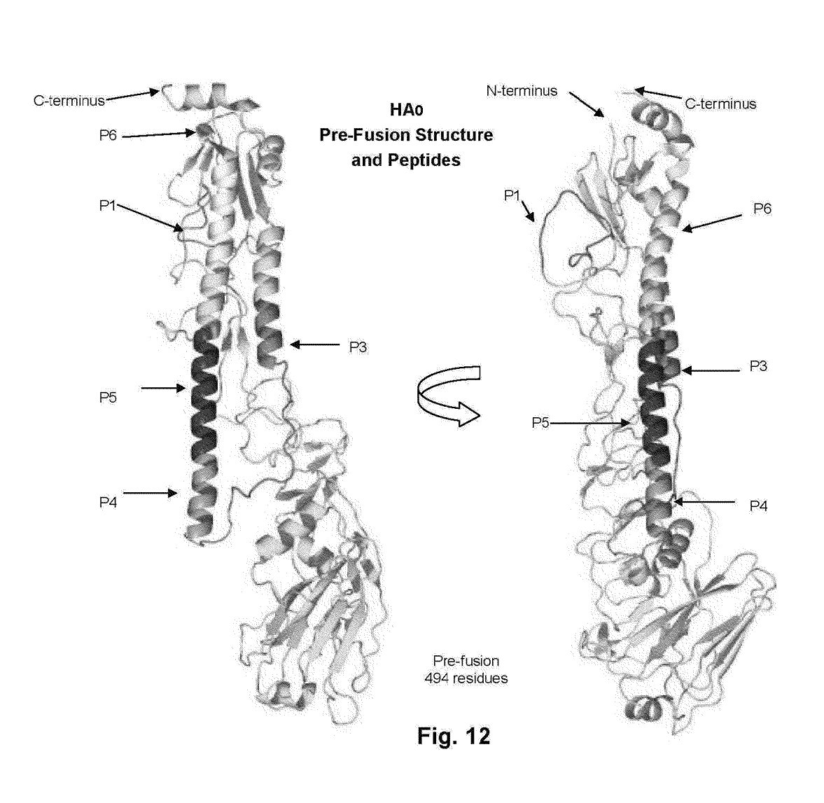

FIG. 13 illustrates the results of influenza challenge studies with rabbit antibody preparations to various influenza peptide immunogens (some data may be duplicated from that in FIG. 7 for immunogens designated 3A, 5A, 6A).

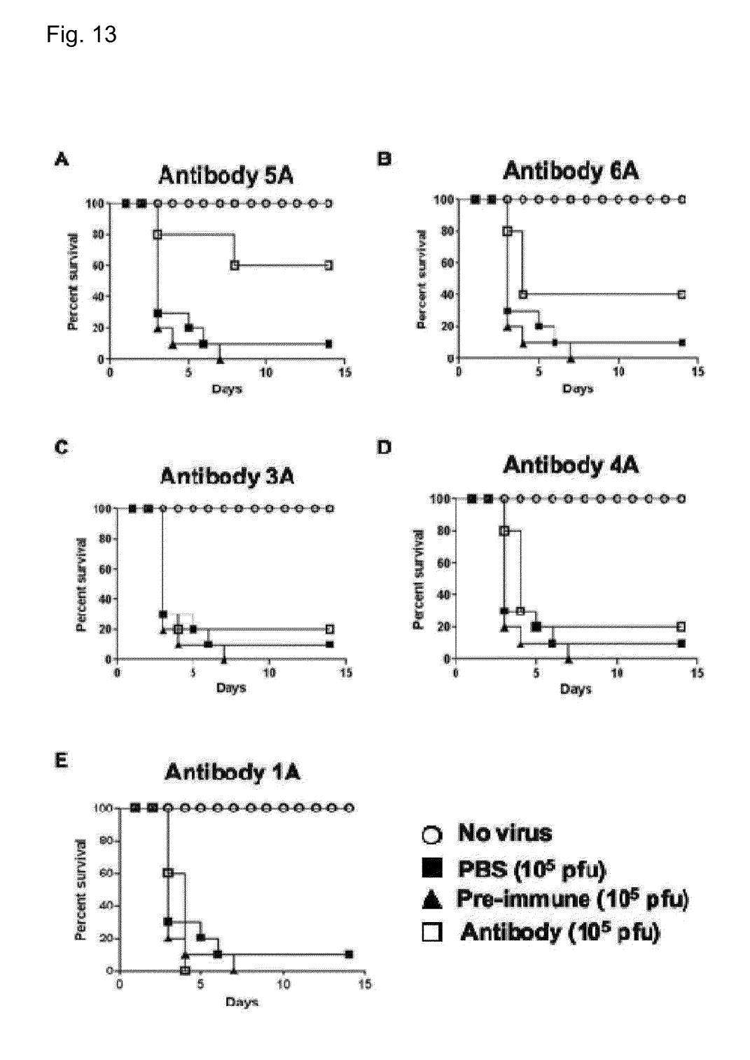

FIG. 14 illustrates examples of hetero two-stranded coiled-coils containing two templated alpha helical different epitopes.

### DETAILED DESCRIPTION OF THE INVENTION

The invention may be further understood by the following non-limiting description.

In one embodiment, the invention comprises a two-stranded peptide conjugate for use in generating an immune response in a subject. By “subject” is meant a vertebrate, such as a bird or mammal, preferably a human. “Individual” is synonymous with “subject.”

As used herein, a “vaccine” is an immunogenic preparation that is used to induce an immune response in individuals. A vaccine can have more than one constituent that is immunogenic. A vaccine can be used for prophylactic and/or therapeutic purposes. A vaccine does not necessarily have to prevent viral infections. Without being bound by theory, the vaccines of the invention can affect an individual's immune response in a manner such that viral infection occurs in a lesser amount (including not at all) or such that biological or physiological effects of the viral infection are ameliorated when the vaccine is administered as described herein.

As used herein, the term “epitope” refers to a molecule (or association of molecules), containing a region capable of eliciting an immune response and/or containing a region capable of specific binding with an antibody. An epitope may be selected, for example, from a portion of a protein not previously known to bind specifically to an antibody.

“Specific binding” refers to binding with a dissociation constant of no greater than about 10⁻⁶ M, preferably no greater than about 10⁻⁷ M, more preferably no greater than about 10⁻⁸ M, still more preferably no greater than about 10⁻⁹M, yet more preferably no greater than about 10⁻¹⁰ M, or alternatively with affinity of at least about 10⁶/M, preferably at least about 10⁷/M, more preferably at least about 10⁸/M, still more preferably at least about 10⁹/M, yet more preferably at least about 10¹⁰/M.

An “effective amount” or a “sufficient amount” of a substance is that amount sufficient to cause a desired biological effect, such as beneficial results, including clinical results, and, as such, an “effective amount” depends upon the context in which it is being applied. In the context of this invention, an example of an effective amount of a vaccine is an amount sufficient to induce an immune response (e.g., antibody production) in an individual. An effective amount can be administered in one or more administrations.

“Stimulation” or “induction” of an immune response can include both humoral and/or cellular immune responses. In one aspect, it refers to an increase in the response, which can arise from eliciting and/or enhancement of a response as compared to the immune response when no vaccine is given at all.

As used herein, and as well-understood in the art, “treatment” is an approach for obtaining beneficial or desired results, including clinical results. For purposes of this invention, beneficial or desired clinical results include, but are not limited to, alleviation or amelioration of one or more symptoms, diminishment of extent of infection, stabilized (i.e., not worsening) state of infection, amelioration or palliation of the infectious state, and decrease in viral titer (whether detectable or undetectable). “Treatment” can also mean prolonging survival as compared to expected survival if not receiving treatment. Symptoms of viral infection (such as influenza infection) is known to one of skill in the art and can include, but is not limited to, fever, coughing, runny nose, congestion, muscle aches, wheezing, nausea, and fatigue.

“Protective immune response” can include any immune response that provides beneficial or desired clinical results. Improving survival rate in an individual can be considered a protective immune response.

### 1. Universal Influenza Vaccines Based on Conformationally-Constrained Peptide Immunogens.

### Introduction and Overview

Influenza is a major global public health challenge. Although several different influenza vaccines and drugs are available to prevent and treat influenza, in the United States alone each year there are 25-50 million cases of influenza and 30,000 to 40,000 deaths. Globally there are approximately 1 billion cases of influenza and up to 500,000 deaths each year. Controlling seasonal influenza A virus is a daunting challenge because of several factors: the virus spreads very rapidly; the incubation period is short; the antigenicity of the viral hemagglutinin (HA) and neuraminidase (NA) glycoproteins continually changes so that new vaccines must be formulated each year; drug resistant mutants are readily selected; high frequency reassortment of genome segments allows transfer of drug resistance or virulence determinants between and among virus strains; and virus infection does not elicit life-long immunity to re-infection.

In addition to seasonal influenza A, pandemics arise sporadically when new virus strains carrying HA and/or NA genes from animals or birds adapt to be capable of transmission from human to human. If the population lacks immunity to the HA and NA antigens of the new virus strains, they may spread around the world with astonishing speed and calamity. Recent examples of zoonotic influenza outbreaks are the H5N1 strain of bird flu, which has a >60% mortality rate in humans but fortunately is not transmitted well from human to human, and the far less virulent H1N1 strain of swine influenza that was first recognized in humans in Mexico in March, 2009. By June, 2009, this H1N1 strain had spread to all 50 states in the United States and at least 73 countries around the globe. To protect against multiple antigenically varying strains of seasonal influenza that co-circulate annually and to provide protection against emerging pandemic strains of influenza A, new approaches to development of influenza vaccines are urgently needed.

We determined that anti-peptide antibodies that recognize conformation-dependent epitopes, particularly in the pre-fusion conformations of class 1 viral fusion glycoproteins including the HA glycoprotein of influenza A, might block the receptor-induced and/or acid pH-induced conformational changes that lead to membrane fusion and virus entry. We tested the hypothesis that immunization with conformation-constrained peptides derived from highly conserved alpha-helical sequences in the stem of influenza A HA would demonstrate the potential to provide long-lasting, broadly cross-reactive protection against many influenza A virus strains.

It is generally difficult or not possible to conduct research on influenza infections in human subjects. Therefore we developed and tested compositions and methods for flu vaccines in a mouse model. We generated conjugate compositions of a stable, synthetic, two-stranded, alpha-helical peptide template that elicits conformation-dependent antibodies to highly conserved helical epitopes in the stem region of the influenza A virus HA glycoprotein.

We explored the ability of the conjugate compositions to induce antibody responses for the capability of providing immune protection against the challenge of infection by influenza which is virulent, indeed fatal, in mice. These experiments proved that antibodies elicited by several novel conformationally-constrained homo two-stranded templated peptide immunogens in fact did protect mice against lethal challenge with a heterotypic influenza A (H1) strain. In embodiments of the invention, these and similar compositions are capable of providing broadly cross-reactive protection against influenza A viruses of different HA types. Thus embodiments of the invention actually fulfill the moniker of a universal vaccine, which can provide immunity against homotypic and heterotypic exposure to influenza infection.

### BACKGROUND

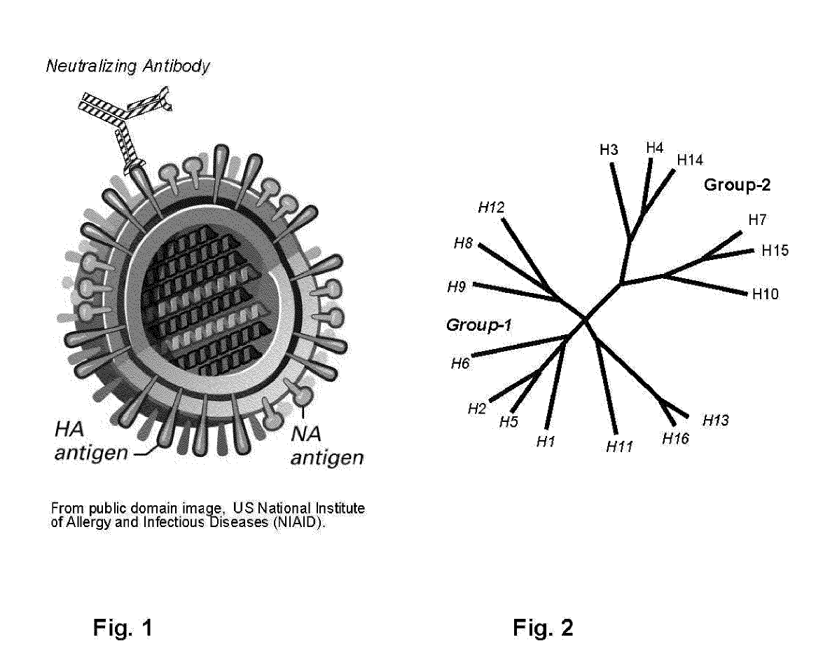

Influenza has an enormous impact on the public health. In an average year, influenza A viruses (FIG. 1) spread rapidly, causing widespread seasonal epidemics of respiratory disease worldwide, which result in at least half a million deaths. Increasing immunity to the virus in the population selects for viruses with mutations in the hemagglutinin (HA) and/or neuraminidase (NA) genes. These mutant viruses can then evade neutralizing antibodies in the population. Such variants may become the predominant strains circulating in the next influenza season. This “antigenic drift” is one important reason why influenza viruses are such successful human pathogens. Immunity to influenza infection depends largely upon antibodies to the viral HA and NA glycoproteins. Infection elicits antibodies that are predominantly virus strain-specific rather than antibodies that are broadly cross-protective against viruses with many different HA and NA proteins. Therefore, each year the vaccine from the previous year becomes obsolete, and influenza vaccines must be adjusted to incorporate HA and NA proteins of the virus strains that are predicted to predominate in the coming flu season. It is very costly to annually develop new vaccines against these predicted epidemic strains, and such vaccines are sometimes poorly matched to the viruses that actually circulate.

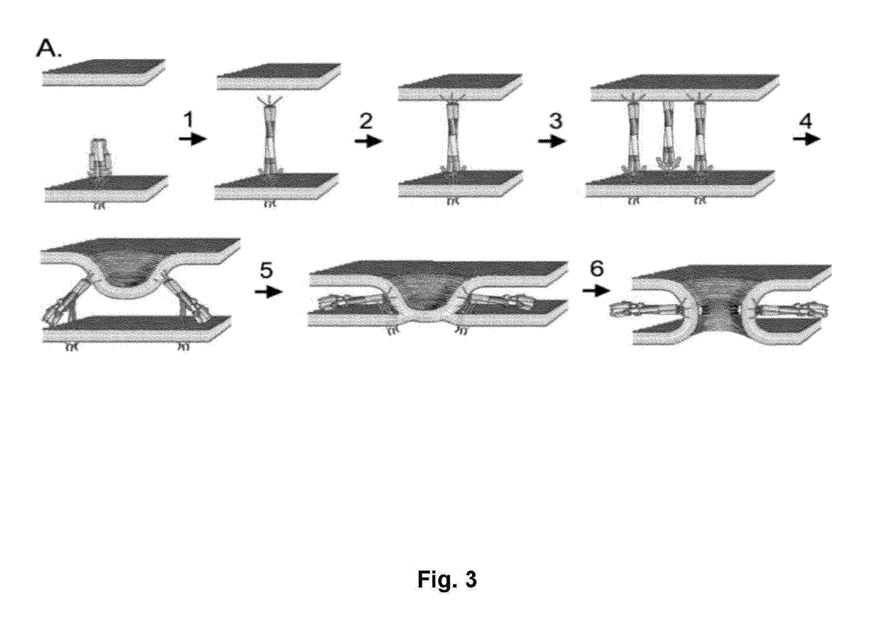

In addition to antigenic drift, antigenically different influenza A virus strains occasionally enter the human population from reservoirs in birds or animals, causing “antigenic shift”, i.e., introduction of an HA or NA type that has generally not previously infected humans. Humans, birds and other mammals harbor influenza A virus strains with at least 16 serologically distinct HA glycoproteins (types H1 through H16) and 9 distinct neuraminidase glycoproteins (N-1-N9). FIG. 2 shows the diversity of HA types in influenza A viruses from humans, birds and mammals. Humans are hosts for types H1, H2 and H3. Sporadically, an influenza A strain from an animal or bird reservoir may infect humans and adapt to permit serial human-to-human transmission, potentially causing a pandemic if the population lacks immunity to these HA and/or NA types.

The annual toll of seasonal influenza on humans is more than a billion cases. In pandemic years, when all humans on earth are susceptible to the new strain of influenza, the number of cases is far higher. The virulence determinants of influenza strains are found in several genome segments. Some zoonotic strains, like swine H1N1 of 2009, are no more virulent than seasonal influenza A strains, while other zoonotic strains are highly virulent, like H5N1 bird flu that kills more than 60% of infected humans but does not spread readily from human to human. Global surveillance coordinated by the World Health Organization aims to detect novel zoonotic influenza virus strains and predict whether or not such new influenza A strains will cause pandemics.

Using the conventional approach, candidate vaccines may be developed to protect against a specific zoonotic virus strain that never becomes widely spread among humans, or antigenic drift in a zoonotic virus may allow it to elude candidate vaccines, realities which emerge after a long process of vaccine design, production, and distribution. Thus, the development and use of new influenza vaccines annually is extremely costly and often inadequately effective. Currently, the single greatest challenge to control of influenza is to develop a universal vaccine that provides long lasting protection against heterotypic strains of influenza A virus.

Several different strategies have been used to try to develop “universal” influenza vaccines that, unlike natural influenza infection, provide broad and long-lasting protection against virus strains with many different HA and NA types. See Subbarao 2006 and Throsby 2008; see also Nabel, G I, Fauci, A S, Induction of Unnatural Immunity Prospects For A Broadly Protective Universal Influenza Vaccine. Nat. Med. 2010 Dec. 16(12):1389-91. Such a vaccine would not have to be changed annually, and would protect against strains changed by antigenic drift as well as against novel zoonotic strains. An effective, broadly protective vaccine would aid in reducing the need for ongoing influenza surveillance in humans, birds and mammals, and similarly decrease or eliminate certain costs associate with changing the vaccine composition each year. Here we describe a novel immunization strategy to elicit broadly cross-protective antibodies against influenza A.

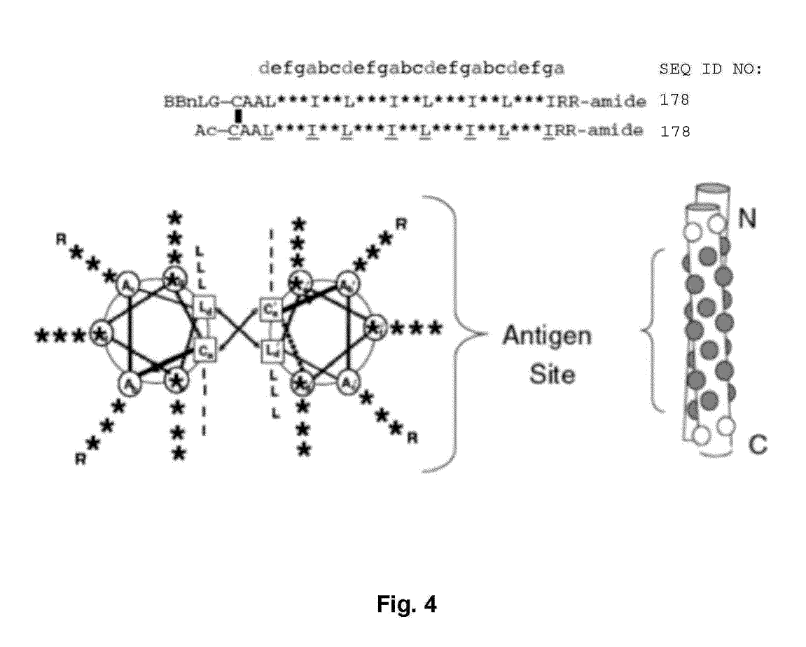

Structure and Functions of Influenza Virus HA Glycoprotein. Influenza HA has an N-terminal domain (called HA₁) that binds to the viral receptor, N-acetyl neuraminic acid, on the cell membrane, and a C-terminal domain (called HA₂) that mediates fusion of the viral envelope with host cell endosomal membranes to initiate infection. The amino acid sequences of HA₁ show considerable variation from one virus strain or HA type to another, while sequences in HA₂ are much more highly conserved. The crystal structure of the ectodomain of HA shows that it is a trimer. Epitopes on the HA₁ domain that elicit strain-specific neutralizing antibodies have been mapped near its membrane distal, receptor-binding domain. The influenza HA₂ domain contains several heptad repeats that undergo a series of massive conformational rearrangements following binding of HA₁ to its receptor at acidic pH (see FIG. 3).

The env glycoprotein of HIV-1, composed of gp120 and gp41, is a Class 1 viral fusion protein like that of influenza HA. Antiviral peptide drugs targeted to the fusion domain of env, such as enfuvirtide (T-20, or trade name Fuzeon) that mimic the C-terminal heptad repeat of HIV-1 gp41, can prevent the conformational changes in the viral glycoprotein and block virus entry and infection. We reasoned that antibodies that target highly conserved regions in the fusion domain of influenza might be able to lock the HA protein in its pre-fusion conformation, preventing membrane fusion.

We incorporated highly conserved alpha-helical sequences in the pre-fusion conformation of the HA₂ domain (the stem of HA) into a highly stable synthetic alpha-helical peptide template that can display two copies of the selected epitope. Alternatively, we note that the sequences in the template may display one or more unique conformation-dependent epitopes mimicking the relevant native conformations relevant to the fusion process. We found that passive immunization with rabbit antibodies to each of 3 different epitopes on the stem of HA partially protected mice from death caused by challenge with 10 LD₅₀ of mouse-adapted influenza A/PR/8/34.

Several research groups investigated whether certain rare human antibodies that bind to HA₂ can prevent the conformational change in the HA₂ domain that is necessary for viral entry into the host cell, and thereby prevent infection of host cells and selection of antibody escape mutants. See Ekiert 2009 and Sui 2009. The antibodies in both of these studies bound to the same alpha-helical domain, called helix A, within the stem of trimeric HA.

Sequence and structural analysis of all HA subtypes reveal two variants of this helix A epitope, corresponding to the two groups of influenza A HA subtypes (FIG. 2). The human antibodies that bind to helix A neutralized influenza A strains with several different HA types, including the 1918 pandemic H1 virus and the H5 bird influenza virus. However, neither these research groups nor others have developed immunogens that can generate antibodies to conformationally-specific epitopes on the HA protein. The present invention in part relates to the significant development of generating templated, homo two-stranded, synthetic peptide immunogens. These immunogens can elicit conformation-specific antibodies to helix A and other alpha-helical epitopes in the HA stem, with the potential to protect against infection with many different influenza virus strains.

Advantages of synthetic peptide vaccines for influenza. Infection with influenza virus or immunization with influenza virus or whole HA protein generates virus strain-specific neutralizing antibodies that predominantly recognize epitopes in the hypervariable regions of the HA₁ domain that surround the receptor binding site. However, a few rare neutralizing human monoclonal antibodies have recently been identified that recognize a highly conserved epitope, Helix A, in the HA₂ domain or stem of HA and interfere with membrane fusion and virus entry. These rare antibodies can neutralize both homotypic and heterotypic influenza strains. Importantly, neither natural infection nor immunization with influenza virus or whole HA protein efficiently raises antibodies to helix A. In contrast, embodiments of immunogenic peptide vaccine materials of the invention are able to target this and other highly conserved antigenic regions of influenza A HA which is associated with neutralization and immune protection.

At present all commercially available human influenza vaccines must be formulated each year to contain HA and NA glycoproteins of three currently circulating strains of influenza A and B. They are produced from viruses grown in embryonated chicken eggs or, recently in Europe, in tissue culture cells. Efficient vaccine production in eggs requires selection of a high yield, reassortant virus strain with the appropriate HA and NA proteins. Live attenuated influenza vaccines (LAIV) consist of infectious virions and are administered by the nasal route, and killed vaccines composed of HA and NA proteins purified from egg-grown virus are administered parenterally. Neither of these vaccines, nor influenza virus infection itself, induces a strong neutralizing antibody response to the helix A region of HA₂.

An attractive alternative to using virions or full-length HA proteins to generate neutralizing antibodies for a specific strain of influenza virus is to immunize with highly conserved, conformation-dependent epitopes, e.g., on the stem of HA, that could elicit neutralizing antibody with broad specificity for other influenza virus strains.

Selected protein segments of interest or small epitopes can be synthesized as peptide immunogens for active vaccination or for preparation of therapeutic monoclonal antibodies against pre-selected epitopes on native HA. Synthetic peptide vaccines have many advantages. One of the main advantages to using synthetic peptides as immunogens is that specific epitopes can be targeted, such as epitopes that are not strongly immunogenic in the native protein. Synthetic peptides are also relatively inexpensive to produce and can be obtained in a highly purified form. A major limitation of synthetic peptide vaccines is that the peptide often lacks a specific conformation found in the intact protein and may not elicit antibodies that recognize the native protein on the viral surface. In the native protein, the conformation of the protein segment of interest is stabilized by secondary and tertiary interactions, but the corresponding peptide alone in solution is typically an unstructured random coil. The generation of antibodies directed toward the surface-exposed regions of a protein using synthetic peptides as immunogens representing surface loops and turns has been successful.

However, peptides representing alpha-helical regions are typically unstructured in solution and unable to generate antibodies that recognize alpha-helices in native proteins. As a result, immunization with a synthetic peptide representing an alpha-helical region can give rise to an antibody that recognizes only a non-native conformation and does not recognize the native protein. Thus, an important aspect in using templated conformationally-stabilized synthetic peptides to generate high affinity, protective antibodies against epitopes within native proteins relies on the ability of the peptide immunogens to mimic the three-dimensional structure of the corresponding B-cell epitope in the native protein. Thus, there is a need for structure-stabilized immunogens for generation of antibodies that recognize a specific conformation, like alpha-helices, of key regions in a native protein. Such proteins include Class 1 viral fusion proteins, for example, influenza virus HA protein.

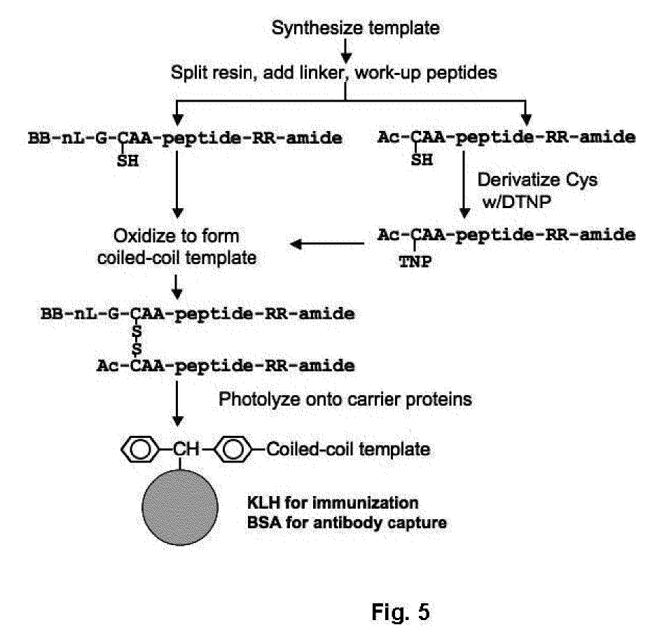

Immunogens for an influenza vaccine. Peptides representing alpha-helical regions of proteins are typically unstructured in solution and do not elicit antibodies that recognize alpha-helices in native proteins. Approximately 50% of all alpha-helices in proteins are amphipathic, i.e. have a polar face and a non-polar face. The non-polar surface of the alpha-helix stabilizes the alpha-helical conformation by hydrophobic interactions within the hydrophobic core of the folded native protein. We developed influenza immunogens with a peptide-based template for generating conformation-specific antibodies that recognize specific alpha-helices in influenza proteins. A preferred embodiment of the influenza vaccine template has a parallel, two-stranded, alpha-helical coiled-coil structure designed to maintain maximum stability through an isoleucine/leucine hydrophobic core and an interchain disulfide bridge. Surface-exposed helical residues from the relevant influenza sequence are inserted into the template (FIG. 4). In an embodiment, a minimum of five out of seven residues in each heptad repeat unit correspond to the alpha-helical sequence selected. The two-stranded template is used for immunization to generate polyclonal antibodies, which are specific not only for the sequence of interest but also for its alpha-helical conformation. This approach is designed to generate specific antibodies targeted against alpha-helical regions of the targeted proteins and epitopes.

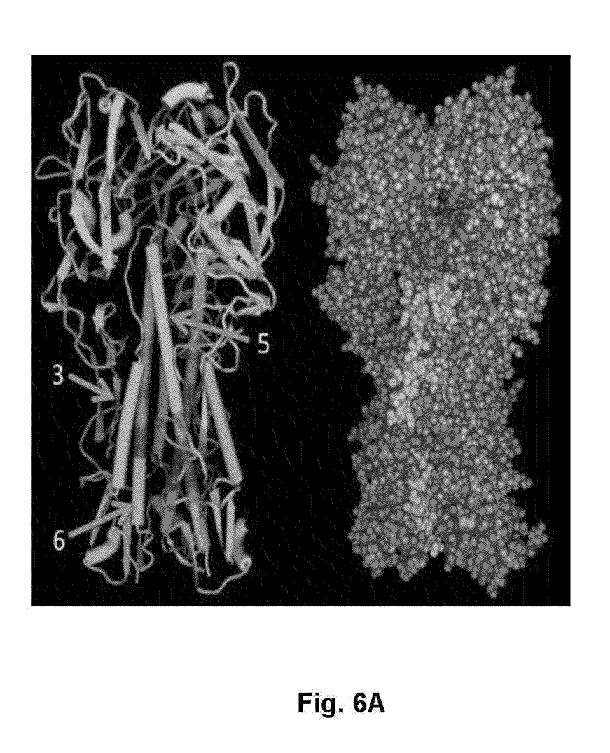

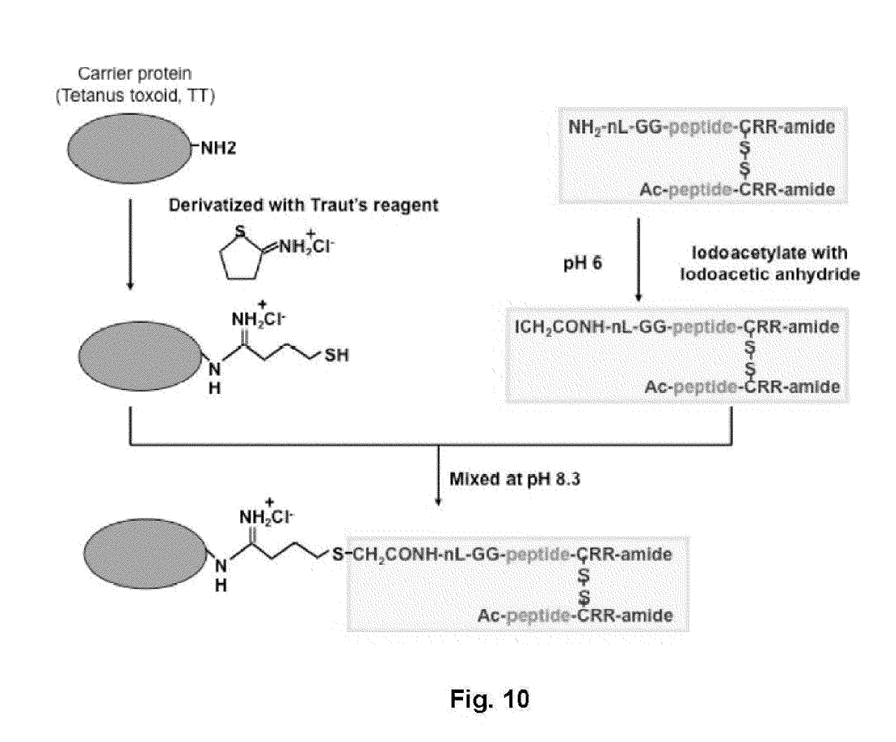

In an embodiment, the templated influenza immunogen is synthesized by solid-phase peptide synthesis, purified, coupled to a carrier protein, and used for immunization. An overview of preparation of the template-carrier protein conjugate for immunization is shown in FIG. 5 and FIG. 10. This method is akin to grafting the molecular surface of the alpha-helix from the influenza protein region of interest into the two-stranded, coiled-coil template.

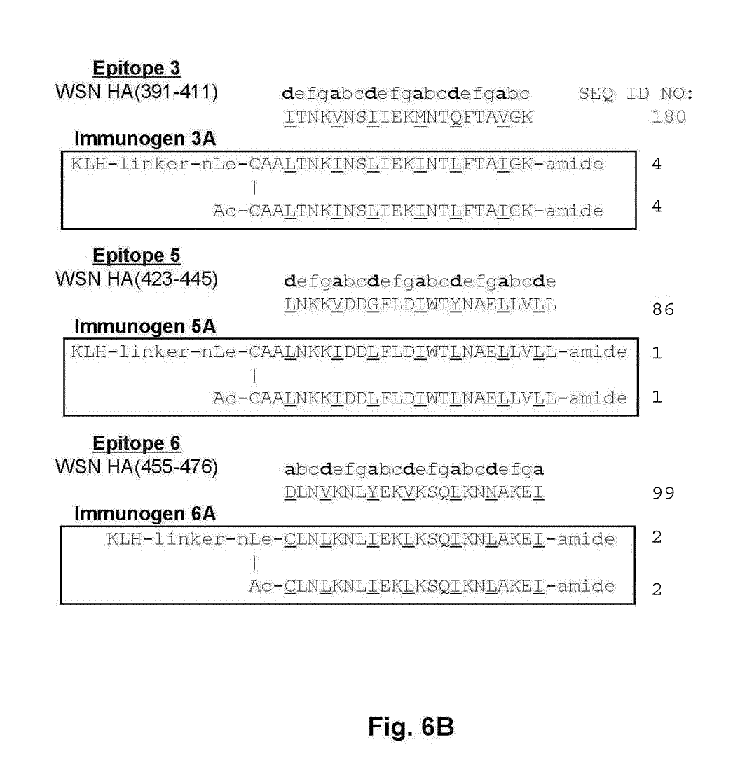

Preparation of Conformationally-Constrained Synthetic Peptide Immunogens in the HA₂ domain of influenza H1N1. Based on the X-ray structure of HA₂, we chose three highly conserved helical regions called epitopes 3, 5 and 6, in the HA₂ or stem domain of HA in its pre-fusion conformation (FIG. 6A), to integrate in two-stranded, alpha-helical, coiled-coil templates (FIG. 6B). These epitopes are exposed on the surface of the space filling model of HA in its prefusion conformation (FIG. 6A, right panel).

The peptide immunogens were prepared by solid-phase synthesis methodology using conventional Fmoc (fluorenylmethoxycarbonyl) chemistry, and two batches were deprotected and N-terminally acylated using either acetic anhydride (Ac) or Fmoc-6-hydrazinonicotinic acid (HNA). The two derivatized peptides were purified by RP-HPLC, and characterized by amino acid analysis and electrospray mass spectrometry (EMS). Preferential disulfide-bridge formation between two different peptide strands (see FIGS. 5a and b) was performed with the control of 2,2′-dithiopyridine. The disulfide-bridged, hetero, two-stranded peptides were purified by RP-HPLC and verified by EMS. Templated two-stranded peptides for immunization were conjugated to keyhole limpet hemocyanin (KLH), and peptides for analysis of peptide-specific anti-sera were conjugated to bovine serum albumin (BSA). Conjugation reactions used the HydraLink coupling Kit, Catalog Nos. 01-63-0121, 01-63-0120 from Novabiochem (Gibbstown, N.J.). The number of peptide molecules per carrier molecule was determined by amino acid analysis using the molar ratio of norleucine (peptide) to phenylalanine (carrier). In general, the concentrations of the conjugates ranged from 0.6 to 0.9 mg/ml at an average peptide to KLH molar ratio of 4:1. The bioconjugation system is shown in Scheme I.

Scheme I for conjugation.

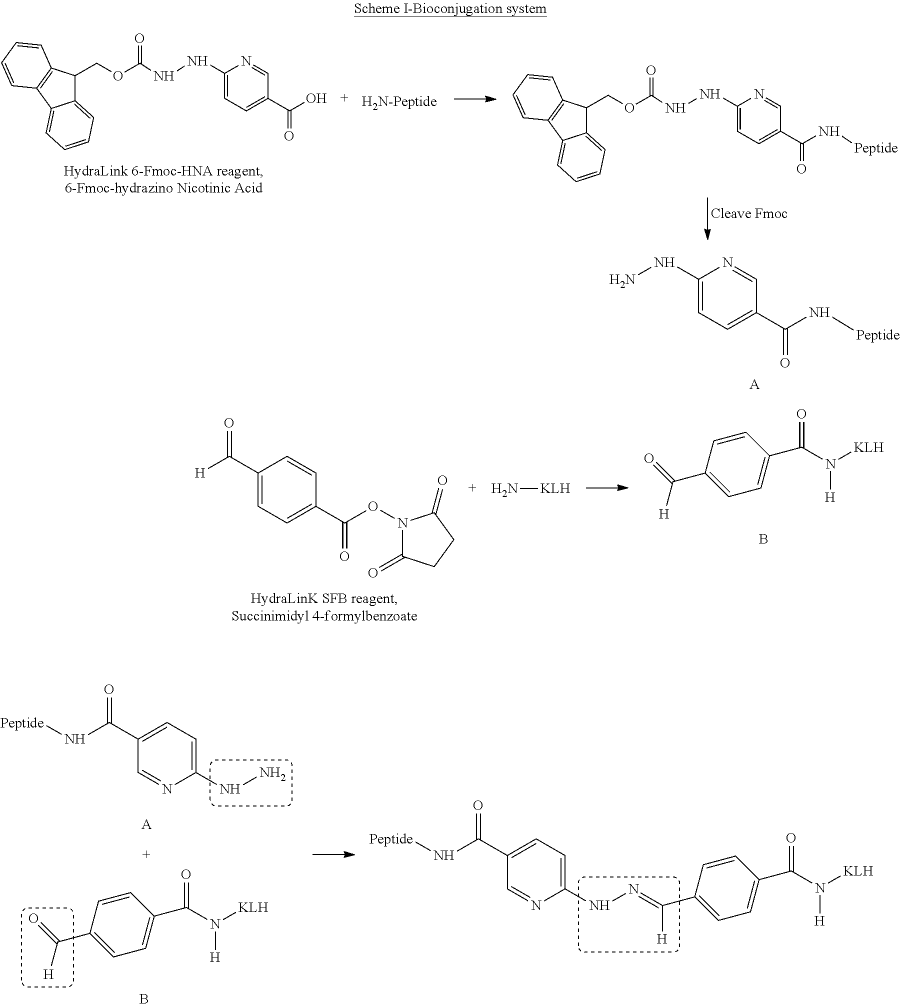

Generation, purification and characterization of anti-peptide antibodies. Briefly, for each immunogen, three New Zealand white rabbits were immunized at two intramuscular sites. Primary doses contained 50 μg of the KLH-peptide conjugate with Freund's complete adjuvant. Boosters at days 7, 28, and 50 contained 50 μg of conjugate, in Freund's incomplete adjuvant. Sera were collected on day 58, and antibodies were purified with protein G affinity chromatography. Enzyme-linked immunosorbent assays (ELISAs) using plates coated with BSA-peptide conjugates showed that these were strong immunogens and that the antibodies had high specificity for their respective coiled-coil templates.

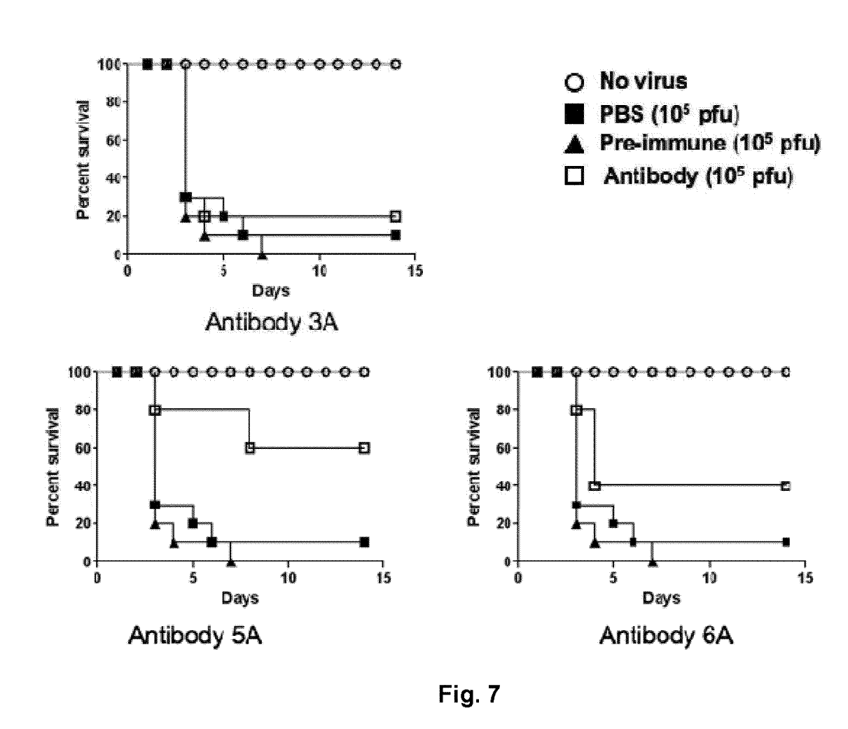

Passive immunization of mice with sera directed against conformation-stabilized, alpha-helical HA₂ peptides and responses to challenge with influenza virus. We next tested whether our rabbit antibodies to conformationally-constrained alpha-helical peptides from influenza HA₂ conferred protection against influenza virus. We used the mouse model of influenza. Ten BALB/c mice were passively immunized by the intraperitoneal route with 1 mg of antibody on days −1, 1 and 3 relative to virus challenge. Control animals received preimmune rabbit antibody, or buffer alone. On day 0, mice were challenged intranasally with 10 LD₅₀ (10⁵ PFU) of mouse-adapted HIN1 influenza A virus, strain PR/8/34 or buffer. Weight change and mortality were monitored daily for 2 weeks. FIG. 7 shows that 9 of 10 of mice treated with PBS and all mice treated with preimmune antibody died by day 7.

In marked contrast, 40% and 60% of mice survived challenge with 10 LD₅₀ of influenza virus after passive immunization with antibodies 6A or 5A, respectively (FIG. 7), to the homo two-stranded conformationally-stabilized alpha-helical HA₂ peptides shown in FIG. 6. Histopathology showed inflammation in lungs of all mice tested that died or were euthanized after loss of more than 15% of body weight as required by protocol. Virus titers were decreased significantly in the lungs of the mice treated by antibodies 5A and 6A.

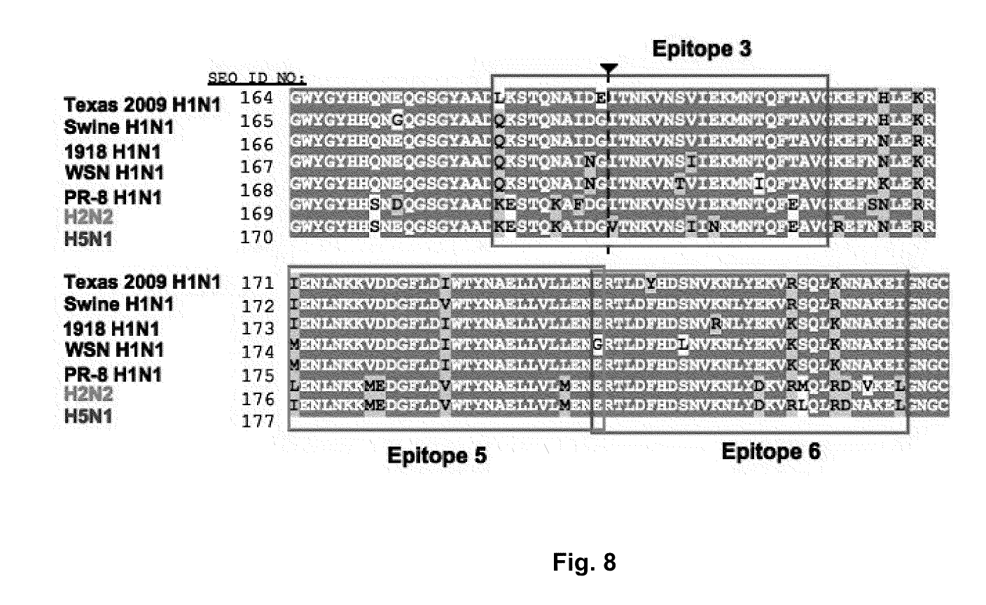

The experiments demonstrated that the peptide-based influenza immunogens were capable of inducing immune responses which generated antibodies that were sufficient upon passive transfer to confer significant protection against challenge with influenza. The antibodies to certain conformationally-stabilized, alpha-helical peptides in the HA₂ domain or stem of HA of the PR/8/34 strain of influenza A H1N1 can protect mice against challenge with the homotypic virus strain. The amino acid sequences of these peptides are relatively conserved among influenza strains (FIG. 8). Thus the anti-peptide antibodies have the potential to also provide protection against heterotypic influenza A viruses. The immunogens may serve as direct vaccine materials for the stimulation of host responses which generate antibodies recognizing conformation-dependent epitopes in the fusion domains of the class 1 viral fusion glycoprotein of influenza hemagglutinin. The vaccinated subjects will therefore be equipped to inhibit influenza virus infection by their antibodies binding to hemagglutinin in its pre-fusion conformation with blocking of the receptor-induced and/or pH-induced conformational changes needed for virus-cell membrane fusion and virus entry. The vaccine material also has the potential to provide cross-protection among various influenza virus strains. Due to the conservation of the epitopes which the peptide-based immunogens mimic, the long term relevance and the efficacy of the immunogens are such that they are able to generate immune responses reflected by antibody levels that remain relatively high, useful, and/or constant in contrast to being more susceptible to reduction by the immune evasion tactics and flexibility of the influenza virus to mutate other regions of the hemagglutinin protein.

In preferred embodiments, tetanus toxoid (TT) is used as a carrier protein for the synthetic peptide immunogens. The carrier TT is compatible for use in human subjects. Keyhole limpet hemocyanin (KLH) can be used in alternate embodiments.

In preferred embodiments, the peptide immunogens are directed to emulate epitopes in alpha-helical regions of the HA₂ or stem domain that are exposed to the surface in the pre-fusion conformation of HA. During influenza infection, the conformational changes in HA that lead to membrane fusion (FIG. 3) generally take place at acid pH within endosomes. The selection of highly conserved, surface-exposed, alpha-helical epitopes in vaccine compositions allow resulting antibodies to bind to these conserved HA epitopes on virions at neutral pH, but may not prevent virus attachment. The protective antibodies can be carried with the virus into the endosomes where they lock the HA in its pre-fusion conformation, block the acid-triggered conformational changes in HA that lead to fusion of the viral envelope with the endosomal membrane, and prevent initiation of virus infection.

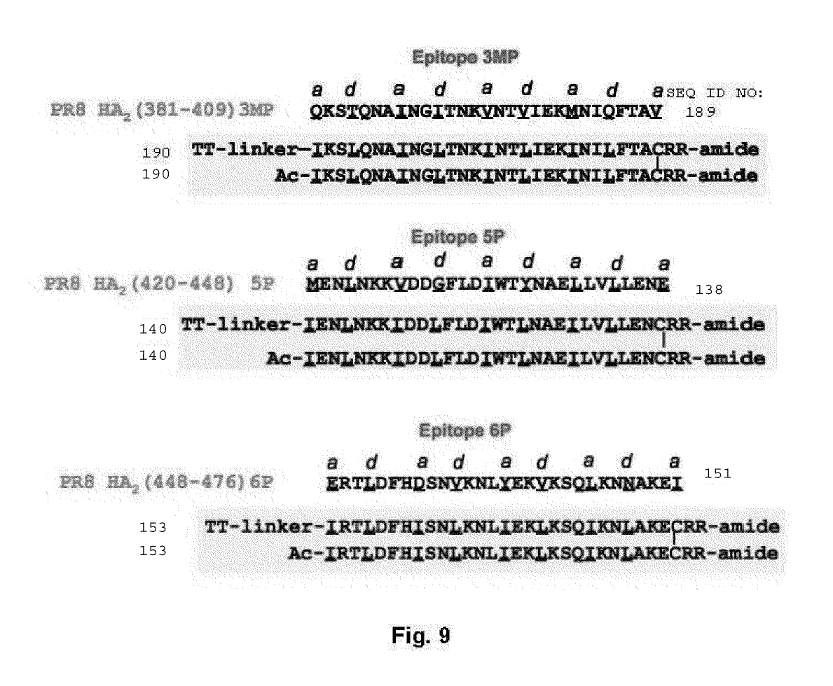

In embodiments, synthetic peptide influenza A vaccines are generated. For peptide design and synthesis, certain conserved alpha-helical epitopes are targeted. These epitopes are exposed on the surface of the HA₂ or stem domain of HA in its pre-fusion conformation. For example, epitopes 5, 6 and 3M (modified to include all of helix A) are used as the basis for design of the peptide immunogens (see FIGS. 8 and 9).

Helix A, amino acids 381-408 of HA, is a particular target for mimicking of the native epitopes of influenza A strains. In data described herein, the peptide 3A (amino acids 391-411) was used as a synthetic HA peptide immunogen to elicit rabbit antibodies. The rabbit serum IgG antibodies showed some capability for conferring immunity when transferred to mice which were infected with a lethal challenge dose of influenza virus; a level of approximately 10-20% protection was observed. Other peptide immunogens are associated with increased efficacy, and still further peptide immunogens are generated with the ability to induce useful immune responses. For example, a homo two-stranded templated conjugated peptide immunogen designated 3 MP (see FIG. 9) is designed to include all of the region of helix A.

The general method for constructing the templated, disulfide-bridged, multi-stranded, coiled-coil peptides is shown in FIG. 5 and FIG. 10. For purposes of illustration, the native helical peptide sequences 3 MP, 5P and 6P (FIG. 9) are templated to form the conformationally-stabilized, coiled-coil immunogens shown in FIG. 9. In the context of heptad repeat units, the residues in positions a and d of the native sequences are replaced with isoleucine and leucine (Ile and Leu), respectively. These templated peptides are synthesized by solid phase methodology as shown in FIG. 5.

The immunogenicity of peptide-based compositions is established or enhanced by covalent coupling to a carrier protein. Although keyhole limpet hemocyanin (KLH) is effective, other carriers such as tetanus toxoid may be more suitable for certain applications, e.g., use in human subjects. An exemplary protocol for coupling the peptide immunogens to TT is shown in FIG. 10. The templated peptides 3 MP, 5P and 6P of mouse-adapted influenza A/PR/8/34 virus HA are linked to the TT carrier protein to create two-stranded, conformation-stabilized, synthetic peptide immunogens.

The iodoacetyl group is introduced to the alpha-amino group of the peptide immunogen, using iodoacetic anhydride at pH6. The carrier protein TT is derivatized with sulfhydryl groups by Traut's agent (2-iminothiolane). This small linker can reduce the likelihood of generation of anti-linker antibodies or additional chemical toxicity. The reaction results in the formation of a stable thioether bond between peptide and carrier TT. The conjugation chemistry is also described by Kao and Hodges (see Chem Biol Drug Des. 2009 July; 74(1):33-42). Other conjugation approaches can be utilized as understood in the art.

The TT-peptide conjugates are used to immunize rabbits and as active vaccines in mice prior to challenge with influenza virus. To screen rabbit antibodies for peptide-specific and conformation-specific antibodies, the two-stranded, coiled-coil HA peptides are coupled to BSA instead of TT. BSA is a convenient adaptor molecule, because it is readily adsorbed to ELISA wells and immobilized on biosensor chips. The amount of each peptide coupled to the carrier proteins is determined by amino acid analysis. By calculating the number of moles of norleucine (nL), which was incorporated in the N-terminal linker of the synthetic peptide, and using the known amino acid composition of TT or BSA, the total amounts of synthetic peptide conjugated to TT or BSA are determined, as well as the molar ratios of synthetic peptide/carrier protein. The ratio of peptide/carrier protein is preferably controlled to be about 6:1.

For rabbit immunization with templated, two-stranded synthetic HA peptide immunogens as shown herein, the immunogenicity of the KLH-conjugated peptides was enhanced by administration with adjuvants (Freund's complete and incomplete). The TT-peptide conjugates are formulated into vaccine compositions using Alhydrogel® (Superfos, Denmark; aluminum hydroxide) as adjuvant, and 0.9% w/v phosphate buffered saline (PBS) as the conjugate vaccine vehicle.

Biophysical studies of the templated HA peptides and native HA peptides. Biophysical studies are conducted to characterize the templated, two-stranded, coiled-coil peptides, for example with respect to the capability for presentation of epitopes in an alpha-helical structure. The structures and stability of peptides for use as vaccines is assessed by circular dichroism (cd) spectroscopy in benign buffer (PBS) and in 50% trifluoroethanol (TFE), and also by thermal denaturation profiles. The oligomerization status of templated peptides is examined by analytical ultracentrifugation analysis and size-exclusion chromatography.

Immunization of rabbits. Briefly, for each TT-peptide conjugate, three rabbits are immunized at two intramuscular sites with 50 ug of conjugate with Alhydrogel® aluminum hydroxide adjuvant. Booster immunizations are performed at days 7, 28, and 50. On day 58, rabbits are euthanized with collection of further samples including blood. Serum IgG is isolated by Protein G affinity chromatography.

The rabbit antibodies against the templated HA peptide immunogens are characterized, for example regarding attributes of peptide-specificity, affinity, and conformation-dependence. Analysis can include the characteristics of whether the antibodies are specific for the immunizing peptide, recognize the alpha-helical conformation of the peptide immunogen, the soluble trimeric influenza HA protein in its pre-fusion and/or post-fusion conformations, and native trimeric influenza HA protein expressed on the cell surface in its pre-fusion conformation.

Enzyme-linked immunosorbent assays (ELISA). To characterize the specificity of the rabbit antibodies for the immunizing peptides, ELISA assays are conducted. The templated, two-stranded, coiled-coil synthetic peptides coupled to BSA are coated on 96 well polystyrene plates. Five percent BSA is used for blocking. Serial 10-fold dilutions in PBS of rabbit IgG antibodies or IgG from rabbit pre-immune sera are incubated with the bound antigens, and bound IgG is detected with goat anti-rabbit IgG coupled to horseradish peroxidase. Each rabbit antibody or normal serum is also tested against immunogens and BSA alone to determine the specificity of the antibodies for the synthetic peptide immunogen. A determination of the immunogenicity of each TT-coupled peptide antigen administered with aluminum hydroxide adjuvant is indicated by the dilution of antibody that gives positive signal in the ELISA. Antibodies that bind with high affinity to helical epitopes on the HA₂ or stem domain of HA have increased relative ability to be carried with virions into endosomes and demonstrate activity to block acid pH-induced conformational changes in HA and prevention of virus entry.

Similarly, ELISAs are performed to determine whether each antibody recognizes only the conformationally-stabilized, two-stranded, coiled-coiled peptide immunogen or both the immunogen and the single-stranded peptide with native epitope sequence. In this assay, the native epitope sequence is coupled to BSA as a single stranded peptide, which will likely be unstructured since it is removed from the native protein. Some high affinity antibodies specific for an alpha-helical epitope may bind to a single-stranded, unstructured peptide antigen by inducing it to assume a helical conformation. For a particular immunogen, some antibodies generated by the immunogen can recognize both it and the native peptide, but others may be specific for the immunogen.

Binding of antibodies to native soluble or anchored trimeric HA protein. The ability of the rabbit antibodies versus pre-immune or naïve rabbit IgG to specifically recognize alpha-helical epitopes in the native pre-fusion conformation of the homotypic influenza HA protein is characterized. This is done by ELISA and/or flow cytometry. The soluble, trimeric ectodomain of influenza A/PR/8/34 HA with a C-terminal flag tag is expressed in human embryonic kidney 293T cells and affinity purified. The soluble HA trimers retain their native pre-fusion conformation if maintained at pH>7.0 and 4° C. ELISA assays are used to compare binding of the induced rabbit antibodies versus normal rabbit IgG to the pre-fusion conformation of the target epitope on the soluble, trimeric native spike ectodomain. The full-length, membrane-anchored, recombinant mouse-adapted influenza A/PR/8/34 HA protein is expressed on 293T cells and tested by flow cytometry for whether the rabbit antibodies can bind to the native homotypic HA protein trimer on the plasma membrane. By pre-treatment of the IgGs with neuraminidase before incubating them with the HA-expressing and control cells, the quality of the assay can be ensured for the aspect that binding of antibody to the HA-expressing cells is not due to recognition by the receptor-binding domain of HA to sialic acid moieties on IgG.

Flow cytometry is used to evaluate the cross-reactivity of anti-peptide antibodies with heterotypic strains of influenza A. The full length, trimeric HA proteins from H1 viruses isolated from humans or swine in different years are expressed on 293T cells and tested for binding to induced antibodies. Flow cytometry is also used to determine if induced antibodies also bind to membrane-anchored HA proteins from H2 and H3 influenza A strains.

Assessment of cross-reactivity of anti-peptide antibodies for soluble HA trimers. Binding parameters are assessed including with respect to diverse H1, H2, H3 and H5 strains of influenza A. The binding affinities of antisera to peptide immunogens with soluble HA trimers from different influenza strains are quantitated using surface plasmon resonance techniques, e.g., with a Biacore biosensor. IgG from immune sera to each of the immunogens or IgG from pre-immune sera is immobilized on the biosensor chip surface. Purified soluble HA trimers from the homotypic A/PR/8/34 strain flows over the immobilized antisera. Sensorgrams are generated to indicate on and off rates of binding and the corresponding affinity constant for a given antibody preparation. Although the alpha-helical sequences targeted by our immunogens are generally highly conserved among influenza A viruses, there are a few conservative and several non-conservative substitutions in epitopes 3 MP, 5P and 6P (FIG. 9). These substitutions are evaluated regarding the ability to affect the binding affinity and neutralization activity of anti-peptide antibodies. Results are compared for the binding affinities of soluble HA trimers from diverse strains of influenza A to the immobilized immune or pre-immune rabbit IgG.

Neutralization assays. The antibodies against the peptide immunogens are tested for neutralization of homotypic and heterotypic influenza A viruses or retrovirus pseudotypes containing homotypic or heterotypic HAs. Neutralization activity is tested for homotypic influenza A/PR/8/34 (H1N1) and heterotypic influenza A strains. A microneutralization assay assesses influenza virus neutralization activities of the rabbit anti-peptide antibodies. In an assay, 100 TCID50 of mouse-adapted influenza A/PR/8/34 incubates 37° C. for 1 hr with equal volumes of 4-fold serial dilutions of antibody (stock IgG concentration, 2 mg/ml). Madin-Darby canine kidney (MDCK) cells are added to each well, and plates are incubated in medium containing trypsin for 18 hours. Virus antigens in acid-alcohol fixed cells are detected by indirect ELISA with a Mab directed against the nucleocapsid protein of influenza A virus. Controls include wells inoculated with medium without virus, cells with virus only without IgG, and virus mixed with dilutions of IgG from pre-immune rabbit sera. The results demonstrate the ability of antibodies to neutralize the infectivity of influenza virus. Combinations of antibody preparations can also be evaluated for neutralization activity. In an embodiment, a combination composition is generated with two or more different antibodies to the peptide-based compounds or conjugates.

Testing is optionally performed for selection of antibody-resistant influenza A/PR/8/34 viruses that have mutant HA proteins. Viruses from the endpoint dilutions of the antibody neutralization experiment are amplified and tested again for neutralization by the same antibody. Viruses with increased resistance to antibody neutralization, if any, can be considered potential antibody escape mutants. The HA genes from such viruses are studied, e.g., by sequencing, to identify mutations relating to resistance to neutralization with antibodies to certain epitopes. Upon identification of antibody escape mutants, further determinations are made regarding whether these viruses can be neutralized with antibody to a different peptide immunogen. The susceptibility of candidate escape mutant viruses to neutralization with antibody to a different epitope is used as a factor in evaluation of applications for antibody cocktails.

Microneutralization assays are also employed for testing induced antibodies against one or more influenza A H1N1 virus strains shown in FIG. 8 including the pandemic H1N1 virus from swine in Mexico and humans in the USA in 2009. The illustrated H1N1 virus strains were isolated from humans or swine in geographically distinct areas over several decades. They therefore show considerable diversity in the neutralization epitopes that surround the receptor-binding domain at the tip at the HA spike. However, FIG. 8 shows that alpha-helical epitopes 3 MP, 5P and 6P in the stem of HA are very highly conserved among H1N1 strains. The neutralization endpoints of each rabbit antibody for different H1N1 viruses can be used as a factor to indicate the extent of potential cross-protection. Antibodies are also tested for the ability to neutralize influenza H2N2 and H3N2 strains isolated from humans in different years. Additional peptide-based compounds, conjugates and compositions are generated for H1 and H2 strains of Group-1 viruses and for H3 strains of Group-2 viruses (see FIG. 2).

The antibodies induced to a given peptide compound/conjugate are evaluated for the ability to block entry of retrovirus pseudotypes containing the HA glycoproteins of zoonotic strains with different HA types, including bird flu H5 cloned from birds or humans in 2009, and H7 and H9 proteins from strains that have caused human infection. Murine retroviruses with HA proteins of different influenza viruses are made. Using pseudotypes containing different HA proteins and beta-galactosidase or luciferase reporter genes, antibody-mediated inhibition of HA-dependent transduction of MDCK cells is assessed. Neutralization of pseudotypes with HA proteins from Influenza B strains is also assessed. Peptide-based immunogens for influenza B epitopes are also generated according to the approach herein for influenza A epitopes.

Passive immunization. Antibody preparations arising from the synthetic compounds/conjugates are tested for efficacy against challenge by influenza virus. Passive immunization was demonstrated with rabbit antibody preparations obtained from immunization with the conformation-stabilized epitopes 5 and 6. Protection at levels of 40% and 60%, respectively, was shown in BALB/c mice challenged intranasally with 10 LD50 units of mouse-adapted influenza A/PR/8/34, the homotypic strain. Antibody to immunogen 3A protected 10-20% of mice in two replicate virus challenge experiments.

The protocol for these in vivo protection studies includes intraperitoneal inoculation at days −1, +1 and +3 relative to virus challenge, with IgG from vaccinated rabbits or from pre-immunization controls. Virus-inoculated animals are observed daily with periodic weighing. Determinations are made for individual subjects or treatment groups (pre-immune versus immune rabbit IgG for a given immunogen) regarding the mean time to death. Titrations are performed for infectious virus in the lungs at days 2 and 4 after virus inoculation along with titration of rabbit IgG in mouse serum at days 2, 4, 6, 8 and 14 for survivors. Examination of histopathology in mouse lungs is conducted at relevant times post-inoculation.

Active immunization. Mice are actively immunized with synthetic peptide immunogens targeted to helical epitopes in the HA₂ or stem domain of HA in its pre-fusion conformation. The degree of protection or susceptibility to challenge with virulent influenza A/PR/8/34 virus is assessed.

Materials and methods. Groups (n=10) of 4-week-old BALB/c mice are immunized intraperitoneally with 100 μl containing 500 μg of aluminum hydroxide gel adjuvant plus either PBS, TT alone as a control, or 10 μg of TT-conjugated peptide 3 MP, 5P, or 6P (corresponding to about 1 μg of peptide). Two or three booster immunizations with the same immunogens are given at 2 week intervals. Blood samples are collected on representative animals from each group just before each boost. Antibody titers to the peptide immunogen are tested by ELISA with the peptide immunogen coupled to BSA. Mouse antibodies are tested in vitro for the ability to neutralize homotypic influenza A/PR/8/34 in microneutralization assays. Animals are challenged by intranasal inoculation with 10 LD50 units (105 TCID50) of influenza A PR/8/34 virus. Animals are monitored daily for 14 days after challenge for survival, weight loss, and clinical presentation. Virus titers in lung are determined on days 2, 4, and 6 after inoculation, and histopathology of lungs is compared in animals immunized with TT-coupled peptide vs. PBS or TT alone.

In an embodiment, a cocktail vaccine is provided, i.e., a combination of peptide-based conjugates. Related methods are provided with use of the combination of antigenic materials for immunization of mammals.

In a particular embodiment, an immunogenic composition herein is administered via intramuscular injection. In a further embodiment, multiple administrations are performed. In an embodiment, an immunogenic composition herein is employed in conjunction with a conventional influenza vaccine, e.g., the seasonal trivalent killed formulation, whether in an integrated fashion or as a distinct component with coordinated administration. In another embodiment, an immunogenic composition is employed in connection with an intranasally-administered modified live influenza vaccine.

In embodiments, the universal vaccines of the present invention are capable of providing broad and/or long-lasting protection from infection with one or more homotypic and heterotypic strains of human and/or zoonotic strains of influenza A, or influenza B or C. Relative to the vaccines in the current state of the art, the vaccines described herein have a lower propensity to become obsolete each year due to antigenic drift or shift and are broadly protective against current and future strains of influenza. Yet another advantage of these novel vaccines is a decreased need for expensive influenza surveillance programs currently used on a global scale to predict emergence of new antigenic types of influenza A virus in humans.

In embodiments, conjugate peptide immunogens, or a combination of such peptide immunogens, are capable of providing stable and broadly protective vaccine formulations with the benefit of lasting protection for the host against multiple influenza strains. In further embodiments, the immunogens can enhance the attributes of current strain-specific influenza vaccines, e.g., with respect to cross-protection. Such novel vaccines also can supplement if not replace the need to develop new specific vaccines each year or more often such as in the case of the recent H1N1 outbreak. In other embodiments, antibodies against these novel immunogens can be administered passively to prevent or treat influenza A virus infection.

### 2. Further Peptide-Based Immunogens.

Peptide-based immunogens are generated as described herein using sequences according to the following tables. Variations of sequences are employed as would be understood in the art and according to the teachings herein.

TABLE 1
Epitope 3

| | | SEQ ID |
|---|---|---|
| Group-Strain | Amino Acid Sequence | NO. |
| Group 1 | | |
| Texas 2009 H1N1 | GYAADLKSTQNAIDEITNKVNSVIEKMNTQFTAVGKEF | 11 |
| Swine H1N1 | GYAADQKSTQNAIDGITNKVNSVIEKMNTQFTAVGKEF | 12 |
| WSN H1N1 | GYAADQKSTQNAINGITNKVNSIIEKMNTQFTAVGKEF | 13 |
| PR8 H1N1 | GYAADQKSTQNAINGITNKVNTVIEKMNIQFTAVGKEF | 14 |
| 1918 H1N1 | GYAADQKSTQNAIDGITNKVNSVIEKMNTQFTAVGKEF | 12 |
| H2N2 | GYAADKESTQKAFDGITNKVNSVIEKMNTQFEAVGKEF | 16 |
| H5N1 | GYAADKESTQKAIDGVTNKVNSIIDKMNTQFEAVGRE | 17 |
| Group 2 | | |
| H10 HA | QAADYKSTQKTIDQVTGKLNRLIEKTNTEFESIESE | 18 |
| H7 HA | TAADYKSTQSAIDQITGKLNRLIDKTNQQFELIDNE | 19 |
| H4 HA | TAADLKSTQAAIDQINGKLNRLIEKTNEKYHQIEKE | 20 |
| H14 HA | TAADLKSTQAAIDQINGKLNRLIEKTNEKYHQIEKE | 20 |
| H3 HA | QAADLKSTQAAIDQINGKLNRLIGKTNEKFHQIEKE | 22 |

TABLE 2
Epitope 5

| | | SEQ ID |
|---|---|---|
| Group-Strain | Amino Acid Sequence | NO. |
| Group 1 | ▾ ▾ | |
| Texas 2009 H1N1 | LEKRIENLNKKVDDGFLDIWTYNAELLVLLENERTLDY | 23 |
| Swine H1N1 | LEKRIENLNKKVDDGFLDVWTYNAELLVLLENERTLDF | 24 |
| WSN H1N1 | LEKRMENLNKKVDDGFLDIWTYNAELLVLLENGRTLDF | 25 |
| PR8 H1N1 | LEKRMENLNKKVDDGFLDIWTYNAELLVLLENERTLDF | 26 |
| 1918 H1N1 | LERRIENLNKKVDDGFLDIWTYNAELLVLLENERTLDF | 27 |
| H2N2 | LERRLENLNKKMEDGFLDVWTYNAELLVLMENERTLDF | 28 |
| H5N1 | LERRIENLNKKMEDGFLDVWTYNAELLVLMENERTLDF | 29 |
| Group 2 | | |
| H10 HA | EYQIGNVINWTKDSITDIWTYQAELLVAMENQ | 30 |
| H7 HA | EQQIGNVINWTRDSMTEVWSYNAELLVAMENQ | 31 |
| H4 HA | EGRIQDLEKYVEDTKIDLWSYNAELLVALENQ | 32 |
| H14 HA | EGRIQDLEKYVEDTKIDLWSYNAELLVALENQ | 32 |
| H3 HA | EGRIQDLEKYVEDTKIDLWSYNAELLVALENQ | 32 |

TABLE 3
Epitope 6

| | | SEQ |
|---|---|---|
| | | ID |
| Group-Strain | Amino Acid Sequence | NO. |
| Group 1 | | |
| Texas 2009 | DYHDSNVKNLYEKVRSQLKNNAKEIGNGCF | 35 |
| H1N1 | | |
| Swine H1N1 | DFHDSNVKNLYEKVRSQLRNNAKEIGNGCF | 36 |
| WSN H1N1 | DFHDLNVKNLYEKVKSQLKNNAKEIGNGCF | 37 |
| PR8 H1N1 | DFHDSNVKNLYEKVKSQLKNNAKEIGNGCF | 38 |
| 1918 H1N1 | DFHDSNVRNLYEKVKSQLKNNAKEIGNGCF | 39 |
| H2N2 | DFHDSNVKNLYDKVRMQLRDNVKELGNGCF | 40 |
| H5N1 | DFHDSNVKNLYDKVRLQLRDNAKELGNGCF | 41 |
| Group 2 | | |
| H10 HA | DMADSEMLNLYERVRKQLRQNAEEDGKGC | 42 |
| H7 HA | DLADSEMNKLYERVRKQLRENAEEDGTGC | 43 |
| H4 HA | DVTDSEMNKLFERVRRQLRENAEDKGNGC | 44 |
| H14 HA | DVTDSEMNKLFERVRRQLRENAEDQGNGC | 45 |
| H3 HA | DLTDSEMNKLFEKTKKQLRENAEDMGNGC | 46 |

TABLE 4
Peptide 1A, WSN HA (342-360)

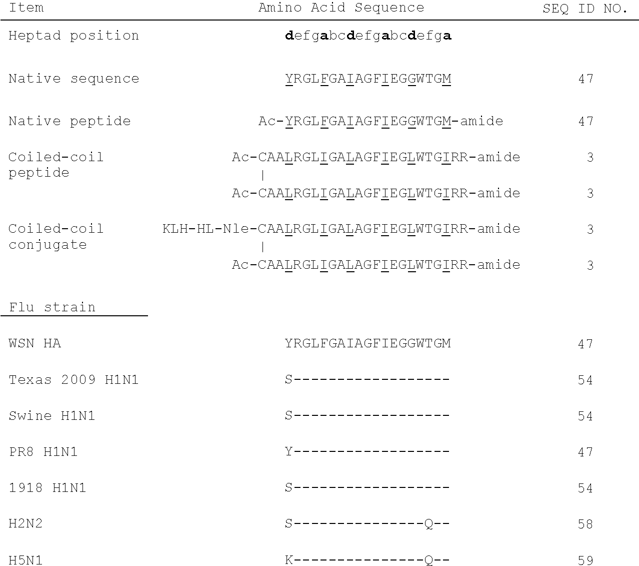

TABLE 5
Peptide 3A, WSN HA (391-411)

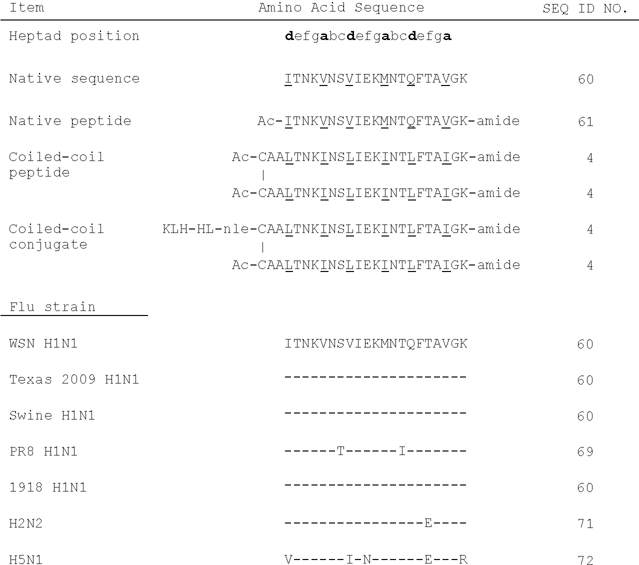

TABLE 6
Peptide 4A, WSN HA (409-429)

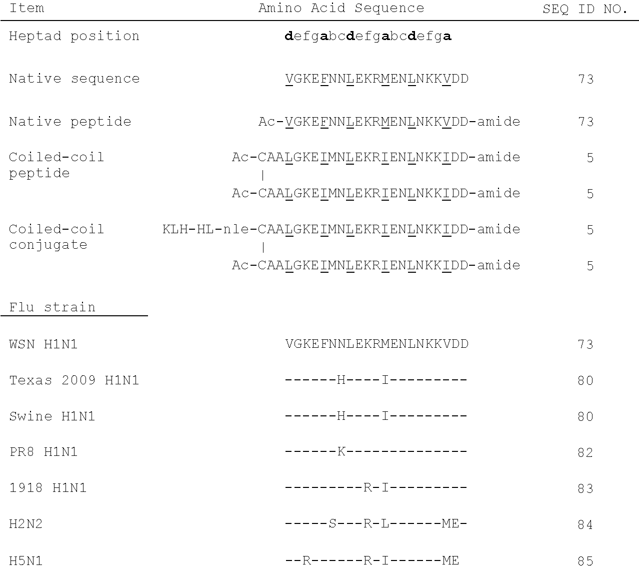

TABLE 7
Peptide 5A, WSN HA (423-445)

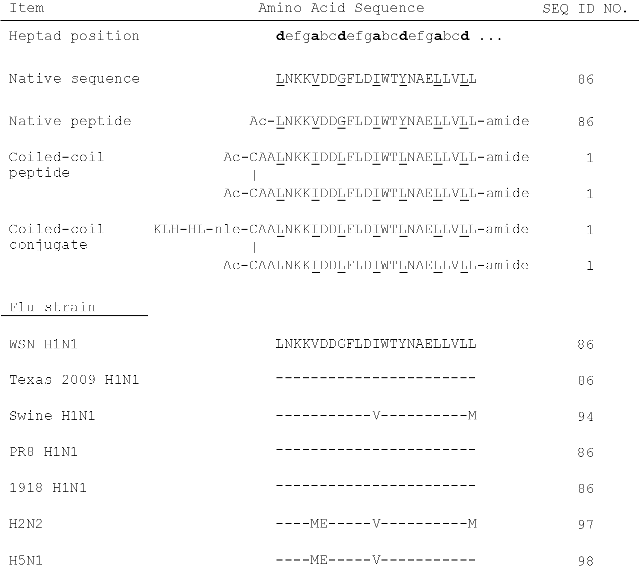

TABLE 8
Peptide 6A, WSN HA (455-476)

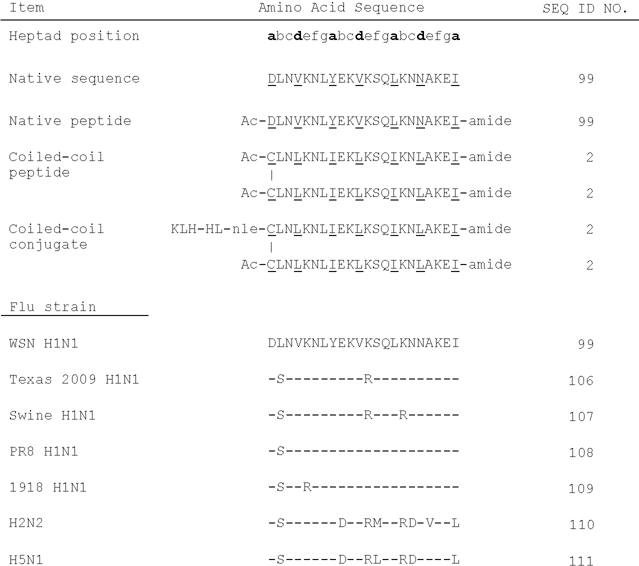

TABLE 9
Peptide 3M* and 3M, WSN HA (381-411)

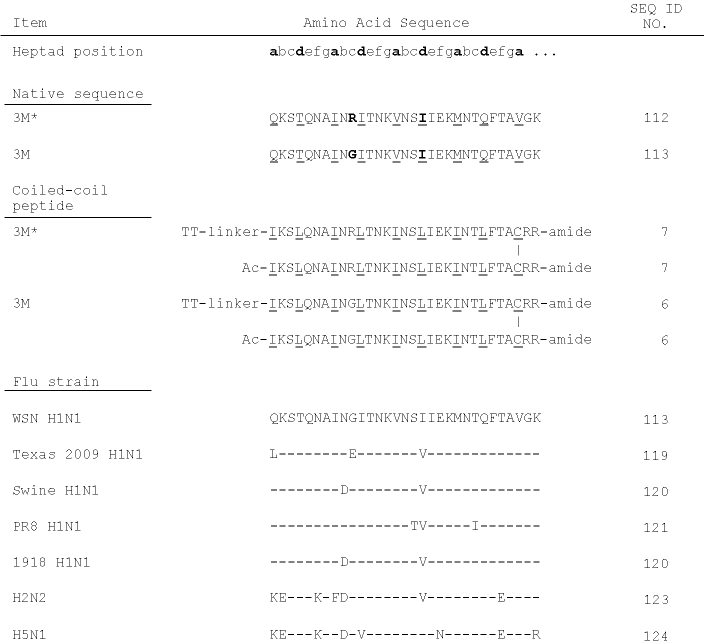

TABLE 10
Peptide 3MP, PR8 HA (381-409)

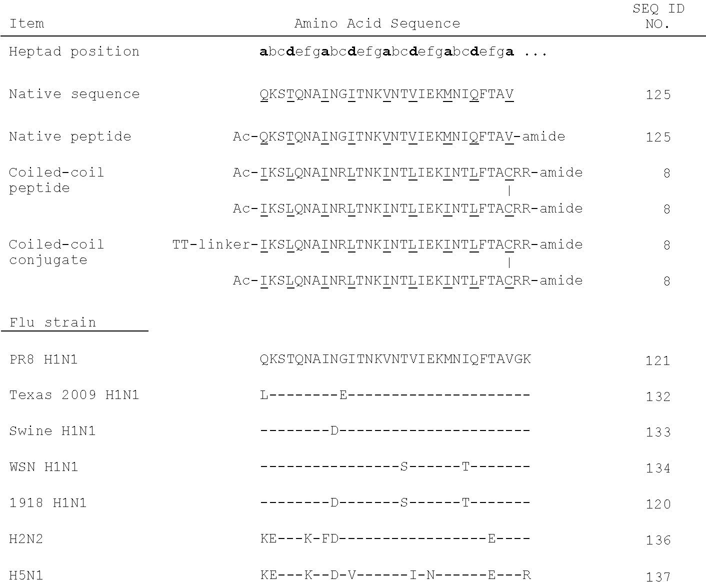

TABLE 11
Peptide 5P, PR8 HA (420-448)

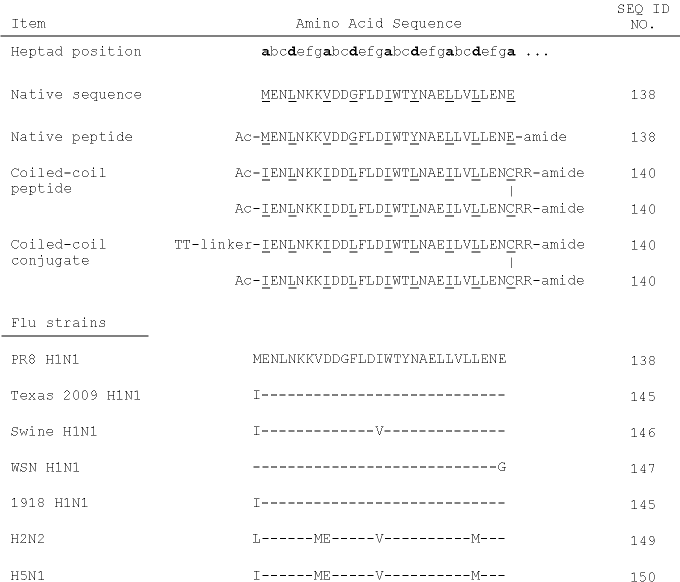

TABLE 12
Peptide 6P, PR8 HA (448-476)

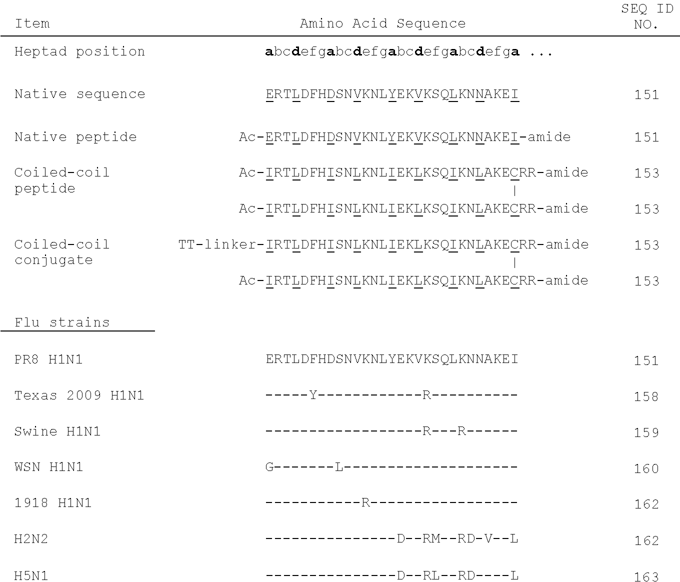

TABLE 13
Epitopes 3, 5 and 6 in Influenza HA. See also FIG. 8.

| | | SEQ ID |
|---|---|---|
| Strain | Amino Acid Sequence | NO. |
| H1N1 | Epitope 3 | |
| Texas 2009 | GWYGYHHQNEQGSCGYAADLKSTQNAIDEITNKVNSVIEKMNTQFTAVGKEFNHLEKR | 164 |
| Swine H1N1 | GWYGYHHQNGQGSCGYAADQKSTQNAIDGITNKVNSVIEKMNTQFTAVGKEFNHLEKR | 165 |
| 1918 H1N1 | GWYGYHHQNEQGSCGYAADQKSTQNAINGITNKVNSIIEKMNTQFTAVGKEFNNLERR | 166 |
| WSN H1N1 | GWYGYHHQNEQGSCGYAADQKSTQNAINGITNKVNTVIEKMNIQFTAVGKEFNNLEKR | 167 |
| PR-8 H1N1 | GWYGYHHQNEQGSCGYAADQKSTQNAIDGITNKVNSVIEKMNTQFTAVGKEFNKLEKR | 168 |
| H2N2 | GWYGYHHSNDQGSCGYAADKESTQKAFDGITNKVNSVIEKMNTQFEAVGKEFSNLERR | 169 |
| H5N1 | GWYGYHHSNEQGSCGYAADKESTQKAIDGVTNKVNSIINKMNTQFEAVGREFNNLERR | 170 |
| H1N1 | Epitope 5 Epitope 6 | |
| Texas 2009 | IENLNKKVDDGFLDIWTYNAELLVLLENERTLDYHDSNVKNLYEKVRSQLKNNAKEIGNGC | 171 |
| Swine H1N1 | IENLNKKVDDGFLDVWTYNAELLVLLENERTLDFHDSNVKNLYEKVRSQLRNNAKEIGNGC | 172 |
| 1918 H1N1 | IENLNKKVDDGFLDIWTYNAELLVLLENERTLDFHDSNVRNLYEKVKSQLKNNAKEIGNGC | 173 |
| WSN H1N1 | MENLNKKVDDGFLDIWTYNAELLVLLENGRTLDFHDLNVKNLYEKVKSQLKNNAKEIGNGC | 174 |
| PR-8 H1N1 | MENLNKKVDDGFLDIWTYNAELLVLLENERTLDFHDSNVKNLYEKVKSQLKNNAKEIGNGC | 175 |
| H2N2 | LENLNKKMEDGELDVWTYNAELLVLMENERTLDFHDSNVKNLYDKVRMQLRDNVKELGNGC | 176 |
| H5N1 | IENLNKKMEDGFLDVWTYNAELLVLMENERTLDFHDSNVKNLYDKVRLQLRDNAKELGNGC | 177 |

Identical residues are indicated as plain text; conserved residues in white text with black background; non-conserved residues underlined (compare with respective items highlighted in green, gray, and white in FIG. 8). SEQ ID NO: designations are indicated at left.

### Additional Embodiments

In embodiments, novel peptide immunogens are generated for protection against influenza. Further vaccines are designed and synthesized. In embodiments, antibody responses and antibodies to the immunogens are characterized and used, including with respect to the characterization of peptide-specificity, affinity, conformation-dependence, structure, and other binding and kinetic parameters. For example, rabbit antibodies raised against the peptide immunogens are assayed for neutralization of homotypic and heterotypic influenza A viruses or retrovirus pseudotypes containing homotypic or heterotypic HAs. Mice are passively immunized with polyclonal rabbit antibody preparations, and the ability to resist challenge is tested. In a particular mammalian animal model, the challenge is performed with homotypic influenza A/PR/8/34 H1N1 virus. Mice are also directly immunized with the synthetic peptide immunogens, and the ability to resist challenge is tested with the challenge strain of virulent influenza A/PR/8/34 H1N1 virus in addition to other virus strains.

Antibodies capable of binding to the peptide immunogens are also further characterized. Candidate antibodies are developed or converted so as to be suitable for therapeutic administration including in human subjects. Techniques for therapeutic antibody-based therapeutics are understood in the art and include such for engineering partially or fully human antibodies and fragments or other antigen recognition molecules.

### Carrier Molecules

Examples of protein carrier molecules include tetanus toxoid (TT), diphtheria toxoid (DT), cholera subunit B, protein D from H. influenza, and bovine serum albumin (BSA). Other carrier molecules including protein and non-protein based carrier molecules are also known in the art.

### Linkers

In one embodiment, linker LX3 is —CH₂—C(═O)— and linker LX2 is -norleucine-glycine-glycine- (-Nle-Gly-Gly-), where the methylene group of LX3 is covalently attached to the carrier, the carbonyl group of LX3 is covalently attached to the amino group of the norleucine residue of LX2, and the C-terminal glycine of LX2 is covalently attached to the N-terminal amino group of PX1. If PX1 is prepared by solid phase peptide synthesis, LX2 can be readily incorporated onto PX1 by extending the synthesis to include Nle-Gly-Gly at the N-terminus of PX1.

When LX3 is —CH₂—C(═O)—, it can be readily incorporated by using iodoacetic acid anhydride to attach an iodoacetyl group, I—CH₂—C(═O)—, to LX2 if LX2 is present, or to the N-terminus of PX1 if LX2 is not present. This yields I—CH2-C(═O)—Nle-Gly-Gly-[PX1], or, if PX2 has been associated with PX1 prior to incorporation of LX3, this yields I—CH2-C(═O)—Nle-Gly-Gly-[PX1-LX1-PX2]. The iodoacetylated complex can then be reacted with a carrier protein containing a nucleophilic moiety, such as a cysteine residue with a free thiol group, resulting in [Carrier protein]-CH2-C(═O)—Nle-Gly-Gly-[PX1-LX1-PX2].

Other linkages that can be used include —OOC—(CH2)ₙ-COO—, where n is an integer from 1 to 12, as LX3, and -Nle-Gly-Gly- as LX2. The compound PG_{acid}-OOC—(CH2)ₙ-COOH, where PG_{acid} is a carboxylic acid protecting group such as t-butyl or benzyl, can be coupled to -Nle-Gly-Gly-[PX1-LX1-PX2] to form PG_{acid}-OOC—(CH2)ₙ-COO-Nle-Gly-Gly-[PX1-LX1-PX2]. The protecting group can then be removed, generating HOOC—(CH2)ₙ-COO-Nle-Gly-Gly-[PX1-LX1-PX2], which can be linked to amino groups on the carrier protein using condensing reagents such as 1-ethyl-3-[3-dimethylaminopropyl]carbodiimide (EDC). Exemplary values for n are n=4 (an adipic acid linker) or n=3 (a glutaric acid linker).

Yet another linker that can be used as LX3 is a maleimide-(CH₂)ₙ-carboxylic acid, of the form:

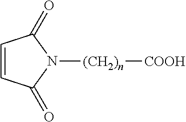

where n is an integer from 1 to 20. These compounds can be readily prepared by reacting a compound of the formula H₂N—(CH2)ₙ-COOH with maleic anhydride, followed by ring closure (see, for example, U.S. Pat. No. 5,360,914). For a LX3-LX2-epitope complex of the form maleimide-(CH₂)ₙ—COO-Nle-Gly-Gly-[PX1-LX1-PX2], reaction with a carrier protein having free thiol (sulfhydryl) groups will result in attachment of the thiol group(s) to the maleimide moiety.

Another linker that can be used as LX3 is benzoylbenzoic acid,

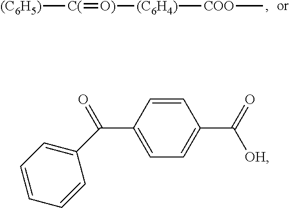

abbreviated as “BB.” This can be readily coupled to -Nle-Gly-Gly-[PX1-LX1-FX2] to form BB-Nle-Gly-Gly-[PX1-LX1-PX2]. The benzophenone moiety is activated via UV light to form the triplet diradical —C.(—O.)—, which can then insert into a C—H bond on the carrier molecule.

Preferably, the linkage from the carrier to the epitope complex is “chemically definite.” That is, LX3, LX2 (if LX3 is not present), or LX3-LX2 (if both are present) is bonded to a specific functional group or groups on the carrier. In this respect, the iodoacetic acid moiety, the dicarboxylic acid moiety, and the maleimide-carboxylic acid moiety will result in a “chemically definite” reaction with the carrier molecule at a specific function group or groups on the carrier molecule, while the BB moiety can incorporate into a variety of functional groups, and is not “chemically definite.”

### Adjuvants

In embodiments, one or more adjuvants are employed in compositions and methods of the invention. In embodiments, an adjuvant is selected as would be understood in the art. Examples of certain adjuvants include Freund's complete adjuvant, incomplete Freund's adjuvant, other oil-water emulsions, and other adjuvants. In preferred embodiments, an adjuvant is compatible for use in human subjects. Another example of an adjuvant is aluminum hydroxide or alum; a particular brand is Alhydrogel® (Superfos, Denmark). Other adjuvants that can be used are those that are approved by the Food and Drug Administration (FDA) for use in humans.

### Other Species

While the description herein emphasizes aspects relating to human influenza strains and applications, embodiments of the invention can also relate to influenza strains and applications for other species. These include in particular equine species and other species of mammals.

### Variants

Variations of certain sequences are provided by this disclosure. One of ordinary skill would understand that the description includes variants according to sequence information and sequences which are related by being at a specified level of relative homology or percent identity. Sequences which are a given percent identical to a reference sequence are provided. This can pertain to sequences of proteins/peptides and nucleic acids. The conditions and parameters for various software and algorithms as understood in the art may be used in establishing the criteria for a given sequence and for a given alignment length.

In an embodiment, the invention provides a peptide compound or conjugate wherein the variation is such that a variant sequence is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identical to a reference sequence. In an embodiment, the variation is a conservative substitution or permits from 1 to 5 changes relative to a reference sequence.

### Synthesis of Peptide Epitopes

The peptide epitopes used in the invention can be prepared by chemical or biological methods known in the art. These methods include solid phase peptide synthesis, solution phase peptide synthesis, fragment condensation (either in solution phase or on solid phase), and recombinant DNA technology.

In one embodiment, the peptide epitopes are synthesized by solid phase peptide synthesis (see Stewart and Young, Solid-Phase Peptide Synthesis, 2^{nd} Ed., Pierce Chemical Co. (Rockford, Ill.), 1984; Merrifield, R. B., 1963, J. Am. Chem. Soc. 85:2149-2154; Fmoc Solid Phase Peptide Synthesis: A Practical Approach (Eds. Chan and White), Oxford University Press (New York), 2000). The peptide epitopes can be synthesized and purified separately, and the peptide epitopes can be associated after synthesis and purification of both epitopes have been completed. Alternatively, the peptide epitopes are synthesized either sequentially or simultaneously by synthesis on a linker which aids in maintaining the association of the peptide epitopes. For example, a branched molecule of the form H2N_{β}—(CH2)-CH(N_{α}H₂)—COOH can be attached via its carboxyl group to a solid-phase synthesis resin, such as a crosslinked benzhydrylamine or methylbenzhydrylamine resin. The α and β nitrogens can be orthogonally protected (such as with a Mtt group and an Fmoc group, an ivDde group and an Fmoc group, or with an Alloc group and Fmoc group), and one chain is synthesized to the desired length, followed by synthesis of the other chain to its desired length. The covalently linked two-stranded peptide is then cleaved from the solid phase resin and purified.

The peptides can have routine modifications, such as acetylation of the N-terminal residue, amidation of the C-terminal residue, or both acetylation of the N-terminal residue and amidation of the C-terminal residue.

### General Methods

The practice of the present invention will employ, unless otherwise indicated, conventional techniques of molecular biology (including recombinant techniques), microbiology, cell biology, biochemistry, nucleic acid chemistry, and immunology, which are within the skill of the art. Such techniques are explained fully in the literature, such as, Molecular Cloning: A Laboratory Manual, second edition (Sambrook et al., 1989) and Molecular Cloning: A Laboratory Manual, third edition (Sambrook and Russel, 2001), (jointly and individually referred to herein as “Sambrook”). Oligonucleotide Synthesis (M. J. Gait, ed., 1984); Animal Cell Culture (R. I. Freshney, ed., 1987); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987, including supplements through 2001); PCR: The Polymerase Chain Reaction, (Mullis et al., eds., 1994); Current Protocols in Immunology (J. E. Coligan et al., eds., 1991); The Immunoassay Handbook (D. Wild, ed., Stockton Press NY, 1994); Bioconjugate Techniques (Greg T. Hermanson, ed., Academic Press, 1996); Methods of Immunological Analysis (R. Masseyeff, W. H. Albert, and N. A. Staines, eds., Weinheim: VCH Verlags gesellschaft mbH, 1993), Antibodies, A Laboratory Manual, (Harlow and Lane, Cold Spring Harbor Publications, New York, 1988); Using Antibodies: A Laboratory Manual (Harlow and Lane, Cold Spring Harbor Laboratory Press, Cold Spring Harbor, N.Y., 1999), Current Protocols in Nucleic Acid Chemistry (Beaucage et al. eds., John Wiley & Sons, Inc., New York, 2000); Protocols for Oligonucleotides and Analogs, Synthesis and Properties (Agrawal, ed., Humana Press Inc., New Jersey, 1993), Vaccines (Plotkin and Orenstein, eds., 4th ed. 2004); and Vaccines (S. Plotkin, 3rd ed. 1999).

### Methods of Using Conjugates

Templated conjugates of the invention can be used in various ways. In one aspect, the templated conjugates can be used as a vaccine or immunogenic composition to enhance an individual's immune response (e.g., antibody response). The enhanced immune response is relative to what an individual's immune response would be without exposure to the conjugate. In another aspect of the invention, the conjugates can be used to induce an immune response (e.g., antibody response) in the individual being given the conjugate. For example, an individual's antibody response can be enhanced or induced by generating a greater quantity of antibody and/or antibodies that are more effective at neutralizing virus(es) and/or pathogen(s) of interest. The antibody response can also be enhanced or induced by the generation of antibodies that binds with greater affinity to their targets. In some instances, the antibodies generated are capable to binding to viral strain of various subtypes. In other aspects, compositions comprising the conjugates as described herein can be used to increase the number of plasma cells and/or memory B cells that can produce antibodies. Methods for measuring specific antibody responses include enzyme-linked immunosorbent assay (ELISA) and are well known in the art. See, e.g., Current Protocols in Immunology (J. E. Coligan et al., eds., 1991). In some aspects, the administration of the conjugates described herein can induce cytokine production (e.g., IL-4, IL-5, and IL-13) that is helpful for antibody production. Cytokine concentrations can be measured, for example, by ELISA. These and other assays to evaluate the immune response to an immunogen are well known in the art. See, for example, SELECTED METHODS IN CELLULAR IMMUNOLOGY (1980) Mishell and Shiigi, eds., W.H. Freeman and Co, and/or Current Protocols in Immunology (J. E. Coligan et al., eds., 1991).

Accordingly, the conjugates described herein can be considered immunogenic compositions. In one aspect, the conjugates can be a component in an immunogenic composition. In another aspect, the conjugates can be a component in a vaccine composition.

In one aspect, the conjugates described herein are used to induce or enhance an individual's immune response (e.g., antibody production or antibody response) such that the viral infection is reduced and in some cases, inhibited. Reduction of viral infection can be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% from the amount of infection that would have occurred had the immune response not been induced or enhanced. Assays for viral infection are routine and known to one of skill in the art.

In another aspect, the conjugates described herein are used to induce or enhance an individual's immune response (e.g., antibody production or antibody response) such that the viral replication is reduced and in some cases, inhibited. Reduction of viral replication can be at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% from the amount of replication that would have occurred had the immune response not been induced or enhanced. Assays for viral replication are routine and known to one of skill in the art.

### Dosage

The amount of the conjugate, when used as a vaccine, to be administered to an individual in need thereof can be determined by various factors, such as the type of viral infection, the biological and/or physiological response from the individual receiving the vaccine and other factors known to one of skill in the art. As such, the amount of the conjugate to be administered can be adjusted accordingly to achieve the desired beneficial effects. In one aspect, the amount of the conjugate to be used is at least about 1 μg conjugate/kg of the individual. In other aspects, the amount of the conjugate to be used is at least about 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, or 30 μg/kg. In other aspects, the amount of the conjugate to be used is at least about 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg/kg. In other aspects, the amount of the conjugate to be used is about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg conjugate/kg of the individual.

In other aspects, the amount of the conjugate to be used is at most about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg conjugate/kg of the individual. In other aspects, the invention provides for a dosage of range of any of the values given above. For example, the lower limit of the dosage range can be about 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg while the upper limit of the dosage range can be 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, 21 μg/kg, 22 μg/kg, 23 μg/kg, 24 μg/kg, 25 μg/kg, 26 μg/kg, 27 μg/kg, 28 μg/kg, 29 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg or 100 μg/kg.

### Modes of Administration

The conjugates described herein can be administered in various ways. In one aspect, the conjugate is administered as an injectable compound. The injection can be by needle injection or needle-free injection (e.g., jet injection). In another aspect, the conjugate is administered as intranasal delivery. The conjugates can also be administered intramuscularly, subcutaneously, intradermally or some combination of all three. These types of injections are known to one of skill in the art.

### Timing of Administration

The conjugates of the invention can be administered with various timing. Timing can be readily determined by one of skill in the art based on the individual's immune parameters. In one aspect, a one-time administration is contemplated. In other aspects, administering the conjugate more than once is contemplated. In these cases, the conjugate can be administered 2, 3, 4, 5, or more times.

If the conjugate is administered more than once, then the interval between the administrations can be of different duration depending on the need of the individual. In some aspects, the interval between the administrations is about 1, 2, 3, 4, 5, 6, or 7 days. In other aspects, the interval between the administrations is about 8, 9, 10, 11, 12, 13, or 14 days. In other aspects, the interval is about 2.5, 3, 3.5, or 4 weeks. In other aspects, monthly intervals are contemplated. The conjugate can be administered upon a determination of need based on the testing of immune parameters in the individuals or based on symptoms experienced by the individual or the individual's exposure to virus(es) and/or other pathogen(s).

### Pharmaceutical Compositions

The conjugates of the invention can be considered as a pharmaceutical composition and or an immunogenic composition. In addition to the other carriers described herein, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringers or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. The conjugate may also be lyophilized using means well known in the art, for subsequent reconstitution and use according to the invention.

Absorption promoters, detergents and chemical irritants (e.g., keritinolytic agents) can be used to enhance the delivery into a target tissue. For reference concerning general principles regarding absorption promoters and detergents which have been used with success in mucosal delivery of organic and peptide-based drugs, see Chien, Novel Drug Delivery Systems, Ch. 4 (Marcel Dekker, 1992).

Examples of suitable nasal absorption promoters in particular are set forth at Chien, supra at Ch. 5, Tables 2 and 3; milder agents are preferred. Suitable agents for use in the method of this invention for mucosal/nasal delivery are also described in Chang, et al., Nasal Drug Delivery, “Treatise on Controlled Drug Delivery”, Ch. 9 and Table 3-4B thereof, (Marcel Dekker, 1992). Suitable agents which are known to enhance absorption of drugs through skin are described in Sloan, Use of Solubility Parameters from-Regular Solution Theory to Describe Partitioning-Driven Processes, Ch. 5, “Prodrugs: Topical and Ocular Drug Delivery” (Marcel Dekker, 1992), and at places elsewhere in the text.

Pharmaceutical compositions can also include vaccines which are formulated for use to induce an immune response to influenza virus. In one aspect, the invention provides a vaccine comprising two templated alpha helical polypeptides of approximately equal length, wherein each polypeptide comprises at least one heptad repeat, and wherein the two polypeptides have less than about 90% sequence identity; a covalent linkage between the two polypeptides; and a carrier protein covalently linked to one of the polypeptides.

The vaccines can also include a carrier as described here. Examples of carriers which may be used include, but are not limited to, alum, microparticles, liposomes, and nanoparticles.

### Sterility

The conjugates, immunogens, and vaccines can be administered as sterile compositions. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

### Kits

The invention further provides kits (or articles of manufacture) comprising a conjugate of the present invention.

In one embodiment, the invention provides a kit comprising both (a) a composition comprising a conjugate as described herein, and (b) instructions for the use of the composition in a subject. In some embodiments, the instructions are on a label. In other embodiments, the instructions are on an insert contained within the kit.

In another embodiment, the invention provides a kit comprising both (a) a composition comprising a conjugate as described herein; and (b) instructions for the administration of the composition to a subject. In some embodiments, the instructions are on a label. In other embodiments, the instructions are on an insert contained within the kit.

In another embodiment, the invention provides a kit comprising both (a) a composition comprising a conjugate as described herein; and (b) instructions for selecting a subject to which the composition is to be administered. In some embodiments, the instructions are on a label. In other embodiments, the instructions are on an insert contained within the kit.

In another embodiment, the invention provides a kit comprising both (a) at least two compositions, each composition comprising a conjugate as described herein; and (b) instructions for selecting one or more compositions to administer to an individual. In some embodiments, the instructions are on a label. In other embodiments, the instructions are on an insert contained within the kit.

### Nucleic Acids, Polypeptides, and Related Methods

The following terms are used to describe the sequence relationships between two or more nucleic acids or polypeptides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA, gene, or protein sequence, or the complete cDNA, gene, or protein sequence.

(b) As used herein, “comparison window” makes reference to a contiguous and specified segment of a sequence, wherein the sequence in the comparison window may include additions or deletions (i.e., gaps) compared to the reference sequence (which does not include additions or deletions) for optimal alignment of the two sequences. Generally, the comparison window is at least 10 contiguous nucleotides or amino acids in length, and optionally can be 20, 30, 40, 50, 60, 100, or longer. Preferably the comparison window for embodiments of certain peptides is 14, 15, 16, 17, 18, 19, 20, 21, 22, 23, 24, 25, 26, 27, 28, 29, 30, 31, 32, 33, 34, 35, 36, 37, 38, 39, 40, 41, or 42 amino acids in length. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Examples of such mathematical algorithms are the algorithm of Myers and Miller (1988, CABIOS, 4:11); the local homology algorithm of Smith et al. (1981, Adv. Appl. Math., 2:482); the homology alignment algorithm of Needleman and Wunsch (1970, J. Mol. Biol., 48:443); the search-for-similarity-method of Pearson and Lipman (1988, PNAS USA, 85:2444); the algorithm of Karlin and Altschul (1990, PNAS USA, 87:2264), modified as in Karlin and Altschul (1993, PNAS USA, 90:5873). Raghava G P, Barton G J., Quantification of the variation in percentage identity for protein sequence alignments, BMC Bioinformatics. 2006 Sep. 19; 7:415. Raghava G P, Searle S M, Audley P C, Barber J D, Barton G J., OXBench: a benchmark for evaluation of protein multiple sequence alignment accuracy, BMC Bioinformatics. 2003 Oct. 10; 4:47.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988, Gene, 73:237), Higgins et al. (1989, CABIOS, 5:151), Corpet et al. (1988, Nucl. Acids Res., 16:10881), Huang et al. (1992, CABIOS, 8:155), and Pearson et al. (1994, Meth. Mol. Biol., 24:307). The ALIGN program is based on the algorithm of Myers and Miller, supra. The BLAST programs of Altschul et al. (1990, J. Mol. Biol., 215:403; and 1997, Nuc. Acids Res., 25:3389) are based on the algorithm of Karlin and Altschul supra.

Software for performing BLAST analyses is publicly available through the National Center for Biotechnology Information (ncbi.nlm.nih.gov on the World Wide Web). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence can be less than about 0.1, less than about 0.01, or less than about 0.001.

To obtain gapped alignments for comparison purposes, Gapped BLAST (in BLAST 2.0) can be utilized as described in Altschul et al. (1997, Nuc. Acids Res., 25:3389). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g. BLASTN for nucleotide sequences, BLASTX for proteins) can be used. The BLASTN program (for nucleotide sequences) uses as defaults a wordlength (W) of 11, an expectation (L) of 10, a cutoff of 100, M=5, N=−4, and a comparison of both strands. For amino acid sequences, the BLASTP program uses as defaults a wordlength (W) of 3, an expectation (1) of 10, and the BLOSUM62 scoring matrix. See the World Wide Web at ncbi.nln.nih.gov. Alignment may also be performed manually by inspection.

In embodiments for purposes of the present invention, comparison of nucleotide sequences for determination of percent sequence identity to given sequences disclosed herein can be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the alternative program.

As used herein, alkyl groups are monovalent saturated hydrocarbons which can be linear, branched, or cyclic, or a combination thereof. Alkyl groups have the number of carbon atoms specified, e.g., C₁-C₁₂ alkyl groups can have between one and twelve carbon atoms, or, if no number is specified, have about 1 to about 8 carbon atoms. Examples of alkyl groups are methyl, ethyl, n-propyl, isopropyl, cyclopropyl, n-butyl, isobutyl, sec-butyl, t-butyl, cyclobutyl, cyclopropyl-methyl, methyl-cyclopropyl, pentyl, cyclopentyl, hexyl, cyclohexyl, heptyl, cycloheptyl, octyl, and cyclooctyl. The alkyl group can be attached to the remainder of the molecule at any position on the alkyl group where a hydrogen can be removed from the corresponding alkane.

As used herein, heteroalkyl groups are monovalent saturated hydrocarbons which can be linear, branched, or cyclic, or a combination thereof, where one or more of the carbon atoms in the group has been replaced by a heteroatom. Heteroatoms include oxygen (—O—), nitrogen (preferably substituted with C₁-C₈ alkyl, for example, —N(CH₃)—), and sulfur (—S—). Heteroalkyl groups have the number of carbon atoms specified, e.g., C₁-C₁₂ heteroalkyl groups can have between one and twelve carbon atoms, or, if no number is specified, have about 1 to about 8 carbon atoms; the number of heteroatoms is not limited, but is preferably from one to three heteroatoms. An example of a heteroalkyl group is —O—CH₂CH₂—O—CH₂CH₂—O—.

As used herein, hydrocarbyl groups are monovalent saturated or unsaturated hydrocarbons which can be linear, branched, or cyclic, or a combination thereof, but excluding aryl and aromatic systems. Hydrocarbyl groups have the number of carbon atoms specified, e.g., C₁-C₁₂ hydrocarbyl groups can have between one and twelve carbon atoms, or, if no number is specified, have about 1 to about 8 carbon atoms. Examples of hydrocarbyl groups are methyl, ethyl, ethenyl, acetylenyl, n-propyl, isopropyl, cyclopropyl, n-butyl, isobutyl, sec-butyl, t-butyl, cyclobutyl, 1,3-butadienyl, cyclopropyl-methyl, methyl-cyclopropyl, pentyl, cyclopentyl, hexyl, cyclohexyl, heptyl, cycloheptyl, octyl, and cyclooctyl. The hydrocarbyl group can be attached to the remainder of the molecule at any chemically feasible position on the hydrocarbyl group.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide or polypeptide sequence in the comparison window may include additions or deletions (i.e., gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. It should be noted that when two sequences of different length are compared, percent sequence identity is calculated with respect to the length of the shorter sequence.

(e)(i) The term “substantial identity” of polynucleotide sequences means that a polynucleotide includes a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and/or at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or at least 95%.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1 to about 20 degrees C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide includes a sequence with at least 70%, 71%, 72%, 73%, 74%, 75%, 76%, 77%, 78%, 79%, 80%, 81%, 82%, 83%, 84%, 85%, 86%, 87%, 88%, 89%, at least 90%, 91%, 92%, 93%, or 94%, or 95%, 96%, 97%, 98% or 99%, sequence identity to the reference sequence over a specified comparison window. Optimal alignment may be conducted using the homology alignment algorithm of Needleman and Wunsch (1970, supra). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tm can be approximated from the equation of Meinkoth and Wahl (1984, Anal Biochem., 138:267); Tm 81.5° C.+16.6 (log M)+0.41 (% GC)—0.61 (% form)—500/L; where M is the molarity of monovalent cations, % GC is the percentage of guanosine and cytosine nucleotides in the DNA, % form is the percentage of formamide in the hybridization solution, and L is the length of the hybrid in base pairs. Tm is reduced by about 1 degree C. for each 1% of mismatching; thus, Tm, hybridization, and/or wash conditions can be adjusted to hybridize to sequences of the desired identity. For example, if sequences with >90% identity are sought, the Tm can be decreased 10° C. Generally, stringent conditions are selected to be about 5 degree C. lower than the Tm for the specific sequence and its complement at a defined ionic strength and pH. However, severely stringent conditions can utilize a hybridization and/or wash at 1, 2, 3, or 4 degree C. lower than the Tm; moderately stringent conditions can utilize a hybridization and/or wash at 6, 7, 8, 9, or 10 degree C. lower than the Tm; low stringency conditions can utilize a hybridization and/or wash at 11, 12, 13, 14, 15, or 20° C. lower than the Tm. Using the equation, hybridization and wash compositions, and the desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), the SSC concentration can be increased so that a higher temperature can be used. An extensive guide to the hybridization of nucleic acids is found in Tijssen (1993, Laboratory Techniques in Biochemistry and Molecular Biology Hybridization with Nucleic Acid Probes, Part 1, Chapter 2, “Overview of principles of 15 hybridization and the strategy of nucleic acid probe assays,” Elsevier, N.Y.). Generally, highly stringent hybridization and wash conditions are selected to be about 5° C. lower than the Tm for the specific sequence at a defined ionic strength and pH.

An example of highly stringent wash conditions is 0.15 M NaCl at 72° C. for about 15 minutes. An example of stringent wash conditions is a 0.2.times.SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1.times.SSC at 45° C. for 15 minutes. An example low stringency wash for a duplex of, e.g., more than 100 nucleotides, is 66.times.SSC at 40° C. for 15 minutes. For short probes (e.g., about 10 to 50 nucleotides), stringent conditions typically involve salt concentrations of less than about 1.5 M, or about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2.times. (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tm for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37 C, and a wash in 0.1.times.SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1.times. to 2.times.SSC (20.times.SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5.times. to 1.times.SSC at 55 to 60° C.

Thus, embodiments of the invention described herein include compositions and methods using variant peptides and polypeptides.

Embodiments also are drawn to compositions or components of peptides/proteins with substitutions of at least one amino acid residue in the polypeptide. In embodiments, amino acid substitutions falling within the scope of the invention include those that do not differ significantly in their effect on maintaining (a) the structure of the peptide backbone in the area of the substitution, (b) the charge or hydrophobicity of the molecule at the target site, or (c) the bulk of the side chain. In particular embodiments, substitutions do not significantly alter at least one of an alpha-helical and or coiled-coil structure or propensity to form such structure.

Naturally occurring amino acid residues are divided into groups based on common side-chain properties: (1) hydrophobic: norleucine, met, ala, val, leu, ile; (2) neutral hydrophilic: cys, ser, thr, asn, gln; (3) acidic: asp, glu; (4) basic: his, lys, arg; (5) residues that influence chain orientation: gly, pro; and (6) aromatic: trp, tyr, phe. Substitution of like amino acids can also be made on the basis of hydrophilicity/hydrophobicity. The hydrophilicity/hydrophobicity scale used in this study is listed as followed: Trp, 33.0; Phe, 30.1; Leu, 24.6; Ile, 22.8; Met, 17.3; Tyr, 16.0; Val, 15.0; Pro, 10.4; Cys, 9.1; His, 4.7; Ala, 4.1; Thr, 4.1; Arg, 4.1; Gln, 1.6; Ser, 1.2; Asn, 1.0; Gly, 0.0; Glu, -0.4; Asp, -0.8 and Lys, -2.0. These hydrophobicity coefficients were determined from reversed-phase chromatography at pH 7 (10 mM PO₄ buffer containing 50 mM NaCl) of a model random coil peptide with a single substitution of all 20 naturally occurring amino acids (see Kovacs, J. M., C. T. Mant and R. S. Hodges. Determination of the intrinsic hydrophilicity/hydrophobicity of amino acid side-chains in peptides in the absence of Nearest-Neighbor or Conformational Effects. Peptide Science (Biopolymers) 84: 283-297 (2006)). We proposed that this HPLC-derived scale reflects the relative difference in hydrophilicity/hydrophobicity of the 20 amino acid side-chains more accurately than previously determined scales (see Mant, C. T., J. M. Kovacs, H. M. Kim, D. D. Pollock and R. S. Hodges. Intrinsic amino acid side-chain hydrophilicity/hydrophobicity coefficients determined by reversed-phase high-performance liquid chromatography of model peptides: comparison with other hydrophilicity/hydrophobicity scales. Peptide Science (Biopolymers) 92: 573-595 (2009)).

One of ordinary skill in the art will appreciate an understanding that a like-for-like substitution approach is contemplated for variants herein, including for certain embodiments where the approach is coupled with the teachings herein.

Exemplary substitutions include those set forth below.

| | Substitutions | |
|---|---|---|
| Original residue | Exemplary | Preferred |
| Ala (A) | ser; gly | ser |
| Arg (R) | lys; his | lys |
| Asn (N) | gln; ser; ala | gln |
| Asp (D) | glu; asn | glu |
| Cys (C) | ser; ala | ser; ala |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu, val |
| Leu (L) | norleucine; ile; val; met; ala; phe | ile, norleu |
| Lys (K) | arg | arg |
| Met (M) | leu; ile; norleu | norleu, leu |
| Phe (F) | leu; val; ile; tyr; trp | tyr |
| Pro (P) | ala | ala |
| Ser (S) | thr | thr |
| Thr (T) | ser | ser |
| Trp (W) | tyr; phe | tyr |
| Tyr (Y) | trp; phe; | phe |
| Val (V) | ile; leu; met; phe; norleucine | ile |

Any sequence listing information is part of the specification and disclosure herewith.

### Synthetic Examples

### Synthetic Example 1

### Disulfide Linkage Between Two Peptides

To form disulfide-bridged peptides, the following procedure is used: 1. Synthesize Peptide 1 (e.g., an acetylated peptide); 2. Cleave and analyze Peptide 1; 3. Purify Peptide 1 by reversed-phase high performance liquid chromatography (RP-HPLC); 4. Analyze fractions, combine and lyophilize; 5. Derivatize Cys of Peptide 1 with DPDT to give Peptide 1 TP; 6. Purify Peptide 1 TP by RP-HPLC; 7. Synthesize Peptide 2 (e.g., can include Nle-G-G linker); 8. Cleave and analyze Peptide 2; 9. Purify Peptide 2 by RP-HPLC; 10. Analyze fractions, combine and lyophilize; 11. Form disulfide bridge Peptide 1 TP and Peptide 2; 12. Purify disulfide bridged Peptide 1-Peptide 2 by RP-HPLC; 13. Analyze fractions, combine and lyophilize; 14. Iodoacetylate the N-terminus of disulfide bridged Peptide 1-Peptide 2; 15. Purify iodoacetylated, disulfide bridged Peptide 1-Peptide 2 by RP-HPLC; 16. Analyze fractions, combine and lyophilize; 17. Conjugate disulfide bridged Peptide 1-Peptide 2 to carrier protein; and 18. Dialyze and lyophilize carrier protein conjugate.

Synthesis of disulfide linker (Optional Linker C) between two cysteine containing peptides. A cysteine-containing peptide is reacted with 2,2′-dithiodipyridine to form the mixed disulfide [Peptide]-S—S-2-pyridine (i.e., [Peptide-S-2-thiopyridine]. The second peptide, containing a free thiol moiety on its cysteine residue, is added to form the disulfide-crosslinked two-stranded peptide (which can be a homo two-stranded conjugate or a hetero two-stranded conjugate).

Step one: the first step of the reaction is carried out with a molar ratio of 1:10 peptide:DTDP. Peptide (e.g., 20 mg) is dissolved in 6 ml reaction solution (3:1 (v/v) acetic acid/H₂O). Ten equivalents of 2,2′-dithiopyridine (DTDP) are added in 100 ul DMF and the reaction is stirred at room temperature for four hours. The reaction can be monitored by LC-MS to detect formation of the peptide-TP product. After the reaction is complete, the reaction mixture is diluted in H₂O, followed by purification by HPLC (e.g. reversed-phase HPLC). The collected fraction(s) from the HPLC are freeze dried to give purified peptide-TP.

Step Two: the peptide-TP product from step one and the second peptide containing a free thiol are dissolved in equimolar amounts in 10 ml 40 mM, NH4Ac, pH 5.5 with 6M GdnHCl. The reaction is incubated at RT for 1 hr. Formation of the two-stranded peptide can be monitored by LC-MS. After the reaction is complete, the two-stranded peptide is purified by HPLC, and the collected fraction(s) are freeze-dried to give the crosslinked two-stranded peptide.

Iodoacetylation of crosslinked two-stranded peptide. Protecting the reagents, reaction, and products from light, iodoacetic anhydride is dissolved in 1,4-dioxane at a concentration of 100 mM. The crosslinked two-stranded peptide is separately dissolved in 100 mM MES, pH 6.0/60% ACN at 0.15 mM. The iodoacetic anhydride solution is slowly added to the peptide solution until reaching the molar ratio 1.2:1, and is incubated at RT for 10 minute. The reaction is monitored by HPLC. After completion, the iodoacetylated is purified by HPLC and lyophilized.

Iodoacetylation can be confirmed by dissolving the iodoacetylated crosslinked two-stranded peptide in 6 M GdnHCl, PBS, pH 8.6, and adding DTT at a concentration of 10 mM. DTT will reduce the disulfide crosslink and also react with the iodoacetyl group. The reaction should yield two peaks when analyzed by LC-MS due to the reduction of the disulfide crosslink, and the masses should correspond to the separate peptides, where the formerly iodoacetylated peptide has the additional mass of the DTT-acetyl group.

Modification of KLH by Traut's reagent to introduce a free thiol group. KLH is dissolved in 1 ml PBS, pH 8.9; 8 M urea, 5 mM EDTA to prepare a 0.1 mM solution of KLH. Traut's reagent is dissolved in water at 4 mg/ml (28 mM). The Traut's reagent is added to KLH solution at molar ratio 1:40. The mixture is incubated for 1 hr at RT, while protecting from light. Unused Traut's reagent is removed using dialysis.

### Conjugation of Iodoacetylated Cross-Linked Two-Stranded Peptide to KLH Modified by Traut's Reagent.

The iodoacetylated cross-linked two-stranded peptide is reacted with the KLH modified by Traut's reagent at a 6:1 two-stranded peptide:KLH ratio in 8 M urea and PBS at RT for up to 48 hours. The progress of the conjugation is followed by reversed-phase HPLC. To terminate the reaction, iodoacetamide in 1 ml water at a concentration of 28 mM is added to the reaction, and the reaction is incubated at RT for 30 min. Dialysis is used to remove free peptide in PBS/8 M urea, 50% ACN/H2O/0.2% TFA. The sample is freeze-dried to yield the salt-free KLH-peptide conjugate.

### Conjugation of Iodoacetylated Cross-Linked Two-Stranded Peptide to BSA Modified by Traut's Reagent.

After preparing solution A of BSA, 68 kD (Traut's reagent modified, 0.2 mM, 8 M urea, PBS), and solution B of iodoacetylated two-stranded peptide (0.5 mM, 8 M urea, PBS), the following reactions are conducted:

reaction X: A:B 1:5, 20 ul A reacts with 40 ul B in 8 M urea, PBS at RT for 1 hr, 4 hrs, and overnight. (RP-HPLC analysis is used to monitor the conjugation); and
reaction R: A:B 1:5, 80 ul A reacts with 160 ul B in 8 M urea, PBS at RT for 1 hr, 4 hrs, and overnight. (RP-HPLC analysis is used to monitor the conjugation). Iodoacetamide in 1 ml water at the concentration of 28 mM is prepared and 100 ul added to the reaction X and R, followed by incubation at RT for 30 min. X and R are combined, dialyzed to remove free peptide in PBS/8 M urea, and then in water/60% ACN/0.2% TFA. Reversed-phase HPLC analysis is used to monitor the removal of free peptide. The sample is freeze-dried to yield salt-free BSA-two-stranded peptide conjugate.

### Synthetic Example 2

### Diaminopropionic Acid Linkage Between Two Peptides

Starting from the following resin-bound diprotected 2,3-diaminopropionic acid reagent:

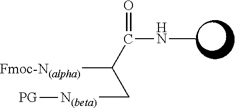

a two-stranded peptide complex cross-linked at the C-terminus can be easily synthesized. The Fmoc group is removed from the alpha-nitrogen of the resin-bound 2,3-diaminopropionic acid and acetylated Peptide 1 is synthesized. After selective deprotection of protecting group PG from the beta nitrogen of the resin-bound 2,3-diaminopropionic acid, Nle-G-G-Peptide 2 is synthesized. Iodoacetylation of the N-terminus of Nle-G-G-Peptide 2 is performed, followed by cleavage of the peptide from the resin. The peptide complex is purified by reversed-phase HPLC, and the fractions are analyzed, combined, and lyophilized. The peptide complex is then conjugated to a carrier protein, followed by dialysis and lyophilization of the carrier protein-peptide complex conjugate.

### STATEMENTS REGARDING INCORPORATION BY REFERENCE AND VARIATIONS

Every compound, composition, formulation, and combination of components described or exemplified herein can be used to practice the invention, unless otherwise stated.

Whenever a range is disclosed herein, e.g., a temperature range, pressure range, time range, composition or concentration range, or other value range, etc., all intermediate ranges and subranges as well as all individual values included in the ranges given are intended to be included in the disclosure. Unless otherwise noted all ranges noted herein are inclusive of the lower and upper range value listed. This invention is not to be limited by the embodiments disclosed, including any shown in the drawings or exemplified in the specification, which are given by way of example or illustration and not of limitation. It will be understood that any subranges or individual values in a range or subrange that are included in the description herein can be excluded from the claims.

All references mentioned throughout this application, for example patent documents including issued or granted patents or equivalents; patent application publications; unpublished patent applications; and non-patent literature documents or other source material; are hereby incorporated by reference herein in their entireties, as though individually incorporated by reference. In the event of any inconsistency between cited references and the disclosure of the present application, the disclosure herein takes precedence. Some references provided herein are incorporated by reference to provide information, e.g., details concerning sources of starting materials, and variations on or additional starting materials, reagents, methods of synthesis, methods of analysis, biological materials, cells, and uses of embodiments of the invention.

All patents and publications mentioned herein are believed to be indicative of the levels of skill of those skilled in the art to which the invention pertains. References cited herein can indicate the state of the art as of their publication or filing date, and it is intended that this information can be employed herein, if needed, to exclude specific embodiments that are in the prior art. For example, when composition of matter are claimed herein, it should be understood that compounds known and properly available in the art prior to Applicant's invention, including compounds for which an enabling disclosure is provided in the references cited herein, are not intended to be included in the composition of matter claims herein.

Any appendix or appendices hereto are incorporated by reference as part of the specification and/or drawings.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof. Thus as used herein, comprising is synonymous with including, containing, having, or characterized by, and is inclusive or open-ended and does not exclude additional, unrecited elements or method steps. As used herein, “consisting of” excludes any element, step, or ingredient, etc. not specified in the claim description. As used herein, “consisting essentially of” does not exclude materials or steps that do not materially affect the basic and novel characteristics of the claim (e.g., relating to an active ingredient). The broad term comprising is intended to encompass the narrower consisting essentially of and the even narrower consisting of. Thus, in any recitation herein of a phrase “comprising one or more claim element” (e.g., “comprising A and B), the phrase is intended to encompass the narrower, for example, “consisting essentially of A and B” and “consisting of” A and B.” Thus, the broader word “comprising” is intended to provide specific support in each use herein for either “consisting essentially of” or “consisting of:” Also, in each instance herein any of the terms “comprising”, “consisting essentially of” and “consisting of” may be replaced with at least either of the other two terms, thereby disclosing separate embodiments and/or scopes which are not necessarily coextensive. An embodiment of the invention illustratively described herein suitably may be practiced in the absence of any element or elements or limitation or limitations not specifically disclosed herein.

The invention has been described with reference to various specific and/or preferred embodiments and techniques. However, it should be understood that many variations and modifications may be made while remaining within the spirit and scope of the invention. It will be apparent to one of ordinary skill in the art that compositions, methods, devices, device elements, materials, procedures and techniques other than those specifically described herein can be employed in the practice of the invention as broadly disclosed herein without resort to undue experimentation. This can extend, for example, to starting materials, biological materials, reagents, synthetic methods, purification methods, analytical methods, assay methods, and biological methods other than those specifically exemplified. All art-known equivalents including functional equivalents of the foregoing (e.g., compounds, compositions, methods, devices, device elements, materials, procedures and techniques, etc.) described herein are intended to be encompassed by this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention may have been specifically disclosed by embodiments, preferred embodiments, and optional features, modification and variation of the concepts herein disclosed may be resorted to by those skilled in the art, and that such modifications and variations are considered to be within the scope of this invention as defined by the appended claims as are or can be set forth in light of the present disclosure.

### REFERENCES

U.S. Pat. No. 7,262,272 Polypeptide compositions formed using a coiled-coil template and methods of use;
U.S. Pat. No. 6,872,806 Polypeptide compositions formed using a coiled-coil template and methods of use
WO/2001/096368, Use of coiled-coil structural scaffold to generate structure-specific peptides
WO/2001/000010, Polypeptide compositions formed using a coiled-coil template and methods of use.
WO/1997/000267, Conformationally-restricted combinatorial library composition and method.
WO/1997/012988, Coiled-coil heterodimers methods and compositions for the detection and purification of expressed proteins.
WO/1995/031480, Heterodimer polypeptide immunogen carrier composition and method.
US 20030021795 A1 Houston, Michael E. et al., Jan. 30, 2003, Use of coiled-coil structural scaffold to generate structure-specific peptides.
U.S. Pat. No. 5,824,483, Houston, Jr., et al., Oct. 20, 1998, Conformationally-restricted combinatiorial library composition and method.
U.S. Pat. No. 5,612,036 Hodges, et al., Mar. 18, 1997, Synthetic Pseudomonas aeruginosa pilin peptide vaccine.
US Pub. No. 20080027006 by Tripet et al., published Jan. 31, 2008 for App. Ser. No. 10/597,914, Compositions And Methods For Modification And Prevention Of Sars Coronavirus Infectivity; WO/2005/077103 publication on Aug. 25, 2005 of International Application No. PCT/US2005/004408 filed Feb. 14, 2005.
Chen J, Skehel J J, Wiley D C, Proc Natl Acad Sci USA. 1999 Aug. 3; 96(16):8967-72. N- and C-terminal residues combine in the fusion-pH influenza hemagglutinin HA(2) subunit to form an N cap that terminates the triple-stranded coiled coil.
Alexander J, Bilsel P, Del Guercio M F, Stewart S, Marinkovic-Petrovic A, Southwood S, Crimi C, Vang L, Walker L, Ishioka G, Chitnis V, Sette A, Assarsson E, Hannaman D, Botten J, Newman M J. Universal influenza DNA vaccine encoding conserved CD4(+) T cell epitopes protects against lethal viral challenge in HLA-DR transgenic mice. Vaccine. 2010 Jan. 8; 28(3):664-72. Epub 2009 Nov. 4.
Fiers W, De Filette M, El Bakkouri K, Schepens B, Roose K, Schotsaert M, Birkett A, Saelens X. Vaccine. 2009 Oct. 23; 27(45):6280-3. M2e-based universal influenza A vaccine.
Ebrahimi S M, Tebianian M, Aghaiypour K, Nili H, Mirjalili A. Mol Biol Rep. epub 2009 Oct. 7. Prokaryotic expression and characterization of avian influenza A virus M2 gene as a candidate for universal recombinant vaccine against influenza A subtypes; specially H5N1 and H₉N₂.
Li O T, Poon L L. Expert Rev Anti Infect Ther. 2009 August; 7(6):687-90. One step closer to universal influenza epitopes.
Kilbourne E D. Nat. Med. 1999 October; 5(10):1119-20. What are the prospects for a universal influenza vaccine?
Adar Y, Singer Y, Levi R, Tzehoval E, Perk S, Banet-Noach C, Nagar S, Amon R, Ben-Yedidia T. Vaccine. 2009 Mar. 26; 27(15):2099-107. Epub 2009 Feb. 13. A universal epitope-based influenza vaccine and its efficacy against H5N1.
Chun S, Li C, Van Domselaar G, Wang J, Farnsworth A, Cui X, Rode H, Cyr T D, He R, Li X. Vaccine. 2008 Nov. 11; 26(48):6068-76. Universal antibodies and their applications to the quantitative determination of virtually all subtypes of the influenza A viral hemagglutinins.
Ng A K, Zhang H, Tan K, Li Z, Liu J H, Chan P K, Li S M, Chan W Y, Au S W, Joachimiak A, Walz T, Wang J H, Shaw P C. FASEB J. 2008 October; 22(10):3638-47. Epub 2008 Jul. 9. Structure of the influenza virus A H5N1 nucleoprotein: implications for RNA binding, oligomerization, and vaccine design.
Denis J, Acosta-Ramirez E, Zhao Y, Hamelin M E, Koukavica I, Baz M, Abed Y, Savard C, Pare C, Lopez Macias C, Boivin G, Leclerc D. Development of a universal influenza A vaccine based on the M2e peptide fused to the papaya mosaic virus (PapMV) vaccine platform. Vaccine. 2008 Jun. 25; 26(27-28):3395-403. Epub 2008 May 12.
Huleatt J W, Nakaar V, Desai P, Huang Y, Hewitt D, Jacobs A, Tang J, McDonald W, Song L, Evans R K, Umlauf S, Tussey L, Powell T J. Vaccine. 2008 January 10; 26(2):201-14. Epub 2007 Nov. 20. Potent immunogenicity and efficacy of a universal influenza vaccine candidate comprising a recombinant fusion protein linking influenza M2e to the TLR5 ligand flagellin.
Leroux-Roels G. Expert Opin Biol Ther. 2009 August; 9(8):1057-71. Prepandemic H5N1 influenza vaccine adjuvanted with AS03: a review of the pre-clinical and clinical data.
U.S. Pat. No. 6,060,065 by (Trimeris/Duke) Barney, et al., issued May 9, 2000 for “Compositions for inhibition of membrane fusion-associated events, including influenza virus transmission.” U.S. Pat. No. 6,068,973; U.S. Pat. No. 7,514,397; U.S. Pat. No. 6,747,126.
PCT International Publication No. WO/2005/044992 by (Tulane) Garry et al., published May 19, 2005 for “Method of preventing virus:cell fusion by inhibiting the function of the fusion initiation region in RNA viruses having Class I membrane fusogenic envelope proteins.” U.S. Pat. No. 7,491,793 for “Influenza virus inhibiting peptides.”
Doherty, P. C., and A. Kelso. 2008. Toward a broadly protective influenza vaccine. J Clin Invest 118:3273-5.
Ekiert, D.C., G. Bhabha, M. A. Elsliger, R. H. Friesen, M. Jongeneelen, M. Throsby, J. Goudsmit, and I. A. Wilson. 2009. Antibody recognition of a highly conserved influenza virus epitope. Science 324:246-51.
Heath, P. T. 1998. Haemophilus influenzae type b conjugate vaccines: a review of efficacy data. Pediatr Infect Dis J 17:S117-22.
Helmke, S. M., S. M. Lu, M. Harmon, J. W. Glasford, T. D. Larsen, S.C. Kwok, R. S. Hodges, and M. B. Perryman. 2006. Myotonic dystrophy protein kinase monoclonal antibody generation from a coiledcoil template. J Mol Recognit 19:215-26.
Howard, M. W., E. A. Travanty, S. A. Jeffers, M. K. Smith, S. T. Wennier, L. B. Thackray, and K. V. Holmes. 2008. Aromatic amino acids in the juxtamembrane domain of severe acute respiratory syndrome coronavirus spike glycoprotein are important for receptor-dependent virus entry and cell-cell fusion. J Virol 82:2883-94.
Lambkin, R., L. McLain, S. E. Jones, S. L. Aldridge, and N. J. Dimmock. 1994. Neutralization escape mutants of type A influenza virus are readily selected by antisera from mice immunized with whole virus: a possible mechanism for antigenic drift. J Gen Virol 75 (Pt 12):3493-502.
Lee, D. L., S. Ivaninskii, P. Burkhard, and R. S. Hodges. 2003. Unique stabilizing interactions identified in the two-stranded alpha-helical coiled-coil: crystal structure of a cortexillin I/GCN4 hybrid coiled-coil peptide. Protein Sci 12:1395-405.
Lu, S. M., and R. S. Hodges. 2002. A de novo designed template for generating conformation-specific antibodies that recognize alpha-helices in proteins. J Biol Chem 277:23515-24.
Rowe, T., R. A. Abernathy, J. Hu-Primmer, W. W. Thompson, X. Lu, W. Lim, K. Fukuda, N. J. Cox, and J. M. Katz. 1999. Detection of antibody to avian influenza A (H5N1) virus in human serum by using a combination of serologic assays. J Clin Microbiol 37:937-43.
Salomon, R., and R. G. Webster. 2009. The influenza virus enigma. Cell 136:402-10.
Subbarao, K., B. R. Murphy, and A. S. Fauci. 2006. Development of effective vaccines against pandemic influenza. Immunity 24:5-9.
Sui, J., W. C. Hwang, S. Perez, G. Wei, D. Aird, L. M. Chen, E. Santelli, B. Stec, G. Cadwell, M. Ali, H. Wan, A. Murakami, A. Yammanuru, T. Han, N. J. Cox, L. A. Bankston, R. O. Donis, R. C. Liddington, and W. A. Marasco. 2009. Structural and functional bases for broad-spectrum neutralization of avian and human influenza A viruses. Nat Struct Mol Biol 16:265-73.
Throsby, M., E. van den Brink, M. Jongeneelen, L. L. Poon, P. Alard, L. Cornelissen, A. Bakker, F. Cox, E. van Deventer, Y. Guan, J. Cinatl, J. ter Meulen, I. Lasters, R. Carsetti, M. Peiris, J. de Kruif, and J. Goudsmit. 2008. Heterosubtypic neutralizing monoclonal antibodies cross-protective against H5N1 and H1N1 recovered from human IgM+ memory B cells. PLoS ONE 3:e3942.
Thompson, B., B. Moesker, J. M. Smit, J. Wilschut, M. S. Diamond, and D. H. Fremont. 2009. A therapeutic antibody against West Nile Virus neutralizes infection by blocking fusion within endosomes. PLoS Pathog 5: e1000453.
Tripet, B., M. W. Howard, M. Jobling, R. K. Holmes, K. V. Holmes, and R. S. Hodges. 2004. Structural characterization of the SARS-coronavirus spike S fusion protein core. J Biol Chem 279:20836-49.
Tripet, B., D. J. Kao, S. A. Jeffers, K. V. Holmes, and R. S. Hodges. 2006. Template-based coiled-coil antigens elicit neutralizing antibodies to the SARS-coronavirus. J Struct Biol 155:176-94.
Wang, T. T., and P. Palese. 2009. Universal epitopes of influenza virus hemagglutinins? Nat Struct Mol Biol 16:233-4.
Wang, W., E. N. Butler, V. Veguilla, R. Vassell, J. T. Thomas, M. Moos, Jr., Z. Ye, K. Hancock, and C. D. Weiss. 2008. Establishment of retroviral pseudotypes with influenza hemagglutinins from H1, H3, and H5 subtypes for sensitive and specific detection of neutralizing antibodies. J Virol Methods 153:111-9.
Yan, Z., B. Tripet, and R. S. Hodges. 2006. Biophysical characterization of HRC peptide analogs interaction with heptad repeat regions of the SARS-coronavirus Spike fusion protein core. J Struct Biol 155:162-75.

1. A conjugate of a conformationally stabilized two-stranded peptide unit and a carrier molecule, the conjugate having the structural formula FX1:
CX-LX3-{[LX2]-[PX1-LX1-PX2]}ₘ (FX1);
wherein
PX1 is a first synthetic peptide,
PX2 is a second synthetic peptide,
LX1 is a first linker for covalently linking the first peptide to the second peptide; wherein
PX1-LX1-PX2 form a conformationally stabilized two-stranded peptide unit;
LX2 is a second linker for linking the two-stranded peptide unit to a carrier molecule,
LX3 is a third linker or direct bond for linking the carrier molecule to the second linker LX2,
CX is an immunogenic carrier molecule, and
m is an integer greater than or equal to one;
wherein the PX1 and PX2 synthetic peptides each independently comprise an adapted peptide sequence corresponding to a stem region of an influenza virus hemagglutinin protein, wherein the adapted sequence consists of a native or synthetic HA₂ domain segment of 15 to 40 amino acids which is integrated in a coiled-coil template, and wherein the conformationally stabilized two-stranded peptide unit, PX1-LX1-PX2 comprises an alpha-helical structure.

2. The conjugate of claim 1 wherein the influenza virus hemagglutinin HA₂ domain segments of first peptide PX1 and second peptide PX2 are each independently a segment derived from the native or synthetic HA₂ domain segment corresponding to at least one of the sequence of amino acid residues for peptides:
P3, amino acids 391-411;
P4, 409-429;
P5, 423-445;
P6, 455-476;
1A, WSN HA (342-360);
3A, WSN HA (391-411);
4A, WSN HA (409-429);
5A, WSN HA (423-445);
6A, WSN HA (455-476);
3M1 [also referred to as 3M], WSN HA (381-411);
3M2 [also referred to as 3M*], WSN HA (381-411);
3 MP, PR8 HA (381-409);
5P, PR8 HA (420-448);
6P, PR8 HA (448-476);
or a variation thereof; where residue numbering is relative to a full sequence for an H3N2 influenza virus strain.

3. The conjugate of claim 1 wherein the adapted peptide sequences for PX1 and PX2 are each independently at least one of the sequences:
5A.T,

| | |
|---|---|
| CAALNKKIDDLFLDIWTLNAELLVLL; | (SEQ ID NO: 1) |
| 6A.T, | |
| CLNLKNLIEKLKSQIKNLAKEI; | (SEQ ID NO: 2) |
| 1A.T, | |
| CAALRGLIGALAGFIEGLWTGIRR; | (SEQ ID NO: 3) |
| 3A.T, | |
| CAALTNKINSLIEKINTLFTAIGK; | (SEQ ID NO: 4) |
| 4A.T, | |
| CAALGKEIMNLEKRIENLNKKIDD; | (SEQ ID NO: 5) |
| 3M1.T/3M.T, | |
| IKSLQNAINGLTNKINSLIEKINTLFTACRR; | (SEQ ID NO: 6) |
| 3M2.T/3M*.T, | |
| IKSLQNAINRLTNKINSLIEKINTLFTACRR; | (SEQ ID NO: 7) |
| 3MP.T, | |
| IKSLQNAINRLTNKINTLIEKINTLFTACRR; | (SEQ ID NO: 8) |
| 5P.T, | |
| IENLNKKIDDLFLDIWTLNAEILVLLENCRR; | (SEQ ID NO: __) |
| 6P.T, | |
| IRTLDFHISNLKNLIEKLKSQIKNLAKECRR; | (SEQ ID NO: __) |

or a variation thereof.

4. The conjugate of claim 2 wherein the variation is such that a variant sequence is at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identical to a reference sequence.

5. The conjugate of claim 2 wherein the variation is a conservative substitution or permits from 1 to 5 changes relative to a reference sequence.

6. The conjugate of claim 1 wherein the coiled-coil template has a peptide sequence with an isoleucine residue at an “a” position and a leucine reside at a “d” position of a heptad helical unit in at least two heptad units corresponding to the HA₂ domain segment.

7. The conjugate of claim 1, wherein the conformationally stabilized two-stranded peptide unit is capable of forming an epitope which mimics an influenza virus structure of a pre-fusion conformation of one or more native hemagglutinin molecules.

8. The conjugate of claim 1, wherein the first and second peptides PX1 and PX2 are each the same peptide.

9. The conjugate of claim 1, wherein the first and second peptides PX1 and PX2 are different peptides.

10. A conformationally stabilized two-stranded peptide compound, the compound having the structural formula FX2:
[PX1-LX1-PX2] (FX2);
wherein
PX1 is a first synthetic peptide,
PX2 is a second synthetic peptide,
LX1 is a first linker for covalently linking the first peptide to the second peptide; wherein PX1-LX1-PX2 form a conformationally stabilized two-stranded peptide unit;
wherein the PX1 and PX2 synthetic peptides each independently comprise an adapted peptide sequence corresponding to a stem region of an influenza virus hemagglutinin protein, wherein the adapted sequence consists of a native or synthetic HA₂ domain segment of 15 to 40 amino acids which is integrated in a coiled-coil template, and wherein the conformationally stabilized two-stranded peptide unit, PX1-LX1-PX2 comprises an alpha-helical structure.

11. The compound of claim 10 further comprising a second linker LX2, where the compound has structural formula FX3:
[LX2]-[PX1-LX1-PX2] (FX3);
wherein
LX2 is a second linker for linking the two-stranded peptide unit to a carrier molecule or a substrate.

12. A composition comprising the conjugate or compound of claim 1 in a pharmaceutically acceptable formulation.

13. A composition comprising the conjugate or compound of claim 1 and an adjuvant.

14. The conjugate of claim 1 wherein the carrier molecule is selected from the group consisting of keyhole limpet hemocyanin (KLH) and tetanus toxoid (TT).

15. A method of inducing an immune response against influenza virus, comprising contacting a mammal with the composition, conjugate or compound of claim 1.

16. A method of reducing an influenza virus infection in an individual in need thereof, comprising administering to the individual an effective amount of the composition, conjugate or compound of claim 1.

17. A molecule which is an antibody, fragment thereof, or other antigen recognition molecule capable of binding to the conjugate or compound of claim 1, wherein the binding is to an epitope of the conformationally stabilized two-stranded peptide unit.

18. The molecule of claim 17 which is humanized or fully human.

19. The molecule of claim 17 which is a monoclonal antibody.

20. The molecule of claim 17 which is part of a polyclonal composition of such molecules.

21. The molecule of claim 17 which is capable of neutralizing an influenza virus.

22. A method of therapy for an influenza infection comprising administering an effective amount of the molecule of claim 17 to a subject in need thereof.

23-25. (canceled)

26. The conjugate or compound of claim 1, wherein the linker LX1 is a disulfide bridge between sulfur-containing amino acid residues of PX1 and PX2.

27. The conjugate or compound of claim 1 wherein the linker LX1 is a compound of the form R₁(—NH₂)—R₂-R₃(—NH₂), where R₁ and R₃ can independently be C₁-C₈ hydrocarbyl, C₁-C₈ alkyl, HOOC—C₁-C₈ hydrocarbyl, or HOOC—C₁-C₈ alkyl, and R₂ can be C₁-C₈ hydrocarbylene (preferably C₁-C₈ alkylene) or a nonentity

28. (canceled)

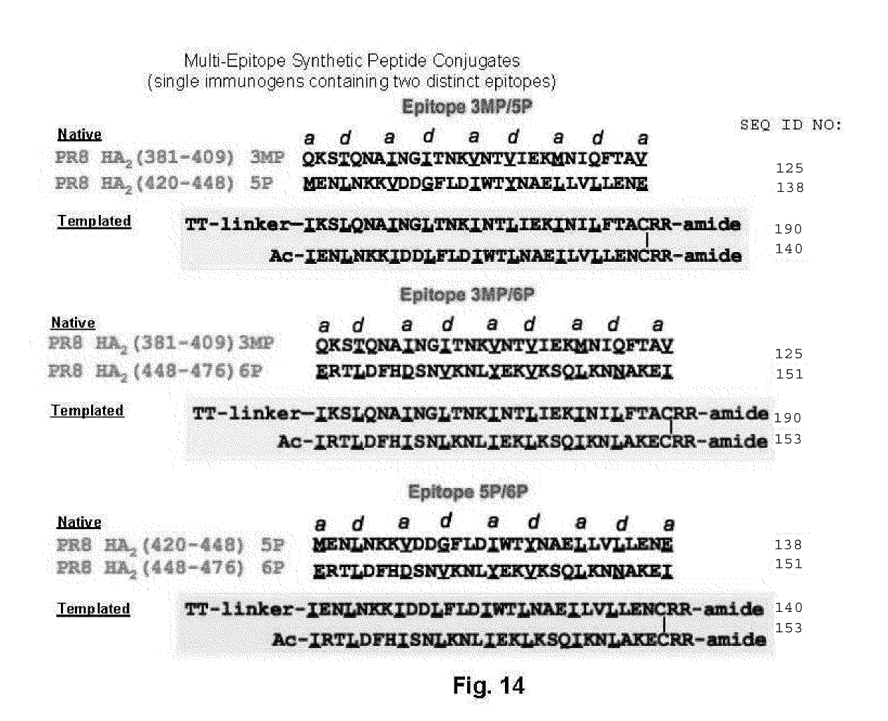

29. The conjugate of claim 9, wherein the first and second peptides PX1 and PX2 are selected from the group consisting of:
a first pair of 3 MP PR8 HA₂ (381-409) (SEQ ID NO: 198) and 5P PR8 HA₂ (420-448) (SEQ ID NO:199);
a second pair of 3 MP PR8 HA₂ (381-409) (SEQ ID NO:202) and 6P PR8 HA₂ (448-476) (SEQ ID NO:203); and
a third pair of 5P PR8 HA₂ (420-448) (SEQ ID NO:206) and 6P PR8 HA₂ (448-476) (SEQ ID NO:207);
wherein for each member of each pair a templated sequence is provided in FIG. 14 and optionally includes a flanking RR component.

30. A method of inducing an antibody response in an individual in need thereof, the method comprising administering the conjugate of claim 1 to an individual in need thereof in an amount sufficient to induce an antibody response in the individual.

31. The method of claim 30 wherein the antibody response is the production of a neutralizing antibody.
